(19) United States
(12) Patent Application Publication
(10) Pub. No.: US 2003/0049680 A1
(43) Pub. Date: Mar. 13, 2003

(54) Methods and compositions utilizing hybrid exact rotamer optimization algorithms for protein design

(76) Inventors: D. Benjamin Gordon, Sommerville, MA (US); Geoffrey K. Hom, Pasadena, CA (US); Stephen Mayo, Pasadena, CA (US); Niles Pierce, Pasadena, CA (US)

Correspondence Address: FLEHR HOHBACH TEST ALBRITTON & HERBERT LLP, Suite 3400, Four Embarcadero Center, San Francisco, CA, 94111-4187, US

(21) Appl. No.: 09/990,769

(22) Filed: Nov. 21, 2001

Related U.S. Application Data
(63) Continuation-in-part of application No. 09/866,511, filed on May 24, 2001.

(51) Int. Cl. G01N 33/53; G06G 7/48; G06G 7/58

(52) U.S. Cl. 435/7.1; 702/19; 703/11

(57) ABSTRACT

The present invention relates to an apparatus and method for quantitative protein design and optimization. In particular, the invention describes the use of Hybrid Exact Rotamer Optimization algorithms in protein design.

This application is a continuing application of U.S. Ser. No. 09/866,511, filed May 24, 2001.

### FIELD OF THE INVENTION

The present invention relates to an apparatus and method for quantitative protein design and optimization. In particular, the invention describes the use of Hybrid Exact Rotamer Optimization algorithms in protein design.

### BACKGROUND OF THE INVENTION

De novo protein design has received considerable attention recently, and significant advances have been made toward the goal of producing stable, well-folded proteins with novel sequences. Efforts to design proteins rely on knowledge of the physical properties that determine protein structure, such as the patterns of hydrophobic and hydrophilic residues in the sequence, salt bridges and hydrogen bonds, and secondary structural preferences of amino acids. Various approaches to apply these principles have been attempted. For example, the construction of α-helical and β-sheet proteins with native-like sequences was attempted by individually selecting the residue required at every position in the target fold (Hecht, et al., Science 249:884-891 (1990); Quinn, et al., Proc. Natl. Acad. Sci USA 91:8747-8751 (1994)). Alternatively, a minimalist approach was used to design helical proteins, where the simplest possible sequence believed to be consistent with the folded structure was generated (Regan, et al., Science 241:976-978 (1988); DeGrado, et al., Science 243:622-628 (1989); Handel, et al., Science 261:879-885 (1993)), with varying degrees of success. An experimental method that relies on the hydrophobic and polar (HP) pattern of a sequence was developed where a library of sequences with the correct pattern for a four helix bundle was generated by random mutagenesis (Kamtekar, et al., Science 262:1680-1685 (1993)). Among non de novo approaches, domains of naturally occurring proteins have been modified or coupled together to achieve a desired tertiary organization (Pessi, et al., Nature 362:367-369 (1993); Pomerantz, et al., Science 267:93-96 (1995)). Though the correct secondary structure and overall tertiary organization seem to have been attained by several of the above techniques, many designed proteins appear to lack the structural specificity of native proteins. The complementary geometric arrangement of amino acids in the folded protein is the root of this specificity and is encoded in the sequence.

Several groups have applied and experimentally tested systematic, quantitative methods to protein design with the goal of developing general design algorithms (Hellinga, et al., J. Mol. Biol. 222: 763-785 (1991); Hurley, et al., J. Mol. Biol. 224:1143-1154 (1992); Desjarlais, et al., Protein Science 4:2006-2018 (1995); Harbury, et al., Proc. Natl. Acad. Sci. USA 92:8408-8412 (1995); Klemba, et al., Nat. Struc. Biol. 2:368-373 (1995); Nautiyal, et al., Biochemistry 34:11645-11651 (1995); Betzo, et al., Biochemistry 35:6955-6962 (1996); Dahiyat, et al., Protein Science 5:895-903 (1996); Jones, Protein Science 3:567-574 (1994); Konoi, et al., Proteins: Structure, Function and Genetics 19:244-255 (1994); Dahiyat and Mayo, Science, 278:82 (1997); Dahiyat and Mayo, Prot. Sci. 5:895 (1996); Gordon and Mayo, J. Comput. Chem. 18:1505 (1998); Dahiyat, et al., Prot. Sci., 6:1333 (1997); Street and Mayo, Foding Des., 3:253 (1998); Gordon et al., Curr. Opin. Struct. Biol., 9:509 (1999)). These algorithms consider the spatial positioning and steric complementarity of side chains by explicitly modeling the atoms of sequences under consideration. To date, such techniques have typically focused on designing the cores of proteins and have scored sequences with van der Waals and sometimes hydrophobic solvation potentials.

Recent studies using coiled coils have demonstrated that core side-chain packing can be combined with explicit backbone flexibility (Harbury et al., PNAS USA 92:8408-8412 (1995); Offer & Sessions, J. Mol. Biol. 249:967-987 (1995). In these cases, the goal was to search for backbone coordinates that satisfied a fixed amino acid sequence.

In addition, the qualitative nature of many design approaches has hampered the development of improved, second generation, proteins because there are no objective methods for learning from past design successes and failures.

Thus, it is an object of the invention to provide computational protein design and optimization via an objective, quantitative design technique implemented in connection with a general purpose computer.

### SUMMARY OF THE INVENTION

In accordance with the objects outlined above, the present invention provides methods executed by a computer under the control of a program, the computer including a memory for storing the program. The methods comprise the steps of receiving a protein backbone structure with variable residue positions, establishing a group of potential rotamers for each of the variable residue positions, wherein at least one variable residue position has rotamers from at least two different amino acid side chains, and analyzing the interaction of each of the rotamers with all or part of the remainder of the protein backbone structure to generate a set of optimized protein sequences. The methods further comprise classifying each variable residue position as either a core, surface or boundary residue. The analyzing step may include a Hybrid Exact Rotamer Optimization (HERO) computation either alone or in combination with a Branch and Terminate (B&T) computation. The HERO computational step may include the use of split singles Dead End Elimination(DEE) or split singles DEE with split flags.

Generally, the analyzing step includes the use of at least one scoring function selected from the group consisting of a Van der Waals potential scoring function, a hydrogen bond potential scoring function, an atomic solvation scoring function, a secondary structure propensity scoring function and an electrostatic scoring function. Additionally, the scoring functions may be modified to emphasize “singles” energy (i.e., rotamer-backbone) terms. The methods further comprise altering the protein backbone prior to the analysis, comprising altering at least one supersecondary structure parameter value. The methods may further comprise generating a rank ordered list of additional optimal sequences from the globally optimal protein sequence. Some or all of the protein sequences from the ordered list may be tested to produce potential energy test results.

In an additional aspect, the invention provides nucleic acid sequences encoding a protein sequence generated by the present methods, and expression vectors and host cells containing the nucleic acids.

In a further aspect, the invention provides a computer readable memory to direct a computer to function in a specified manner, comprising a side chain module to correlate a group of potential rotamers for residue positions of a protein backbone model, and a ranking module to analyze the interaction of each of said rotamers with all or part of the remainder of said protein to generate a set of optimized protein sequences. The memory may further comprise an assessment module to assess the correspondence between potential energy test results and theoretical potential energy data.

### BRIEF DESCRIPTION OF THE DRAWINGS

FIG. 1 illustrates a general purpose computer configured in accordance with an embodiment of the invention.

FIG. 2 illustrates processing steps associated with an embodiment of the invention.

FIG. 3A illustrates processing steps associated with a ranking module used in accordance with an embodiment of the invention. After any DEE step, any one of the previous DEE steps may be repeated. In addition, any one of the DEE steps may be eliminated; for example, original singles DEE need not be run.

FIGS. 3B, 3C and 3D illustrate the processing steps which may comprise HERO.

FIG. 4 is a schematic representation of the minimum and maximum quantities (defined in Equations 24-27) that are used to construct speed enhancements. The minima and maxima are utilized directly to find the (iᵤj_{w})_{mb} pair and for the comparison of the extrema. The differences between the quantities, denoted with arrows, are used to construct the qᵣₛ and qᵤᵥ metrics.

FIGS. 5A, 5B, 5C and 5D depict several super-secondary structure parameters for α/β proteins. The definitions are similar to those previously developed for α/β proteins (Janin & Chothia, J Mol Biol 143:95-128 (1980); Cohen et al., J Mol Biol 156:821-862 (1982)). The helix center is defined as the average C_{α} position of the residues in the helix. The helix axis is defined as the principal moment of the C_{α} atoms of the residues in the helix. (Chothia et al., Proc Natl Acad Sci USA 78:4146-4150 (1981); J Mol Biol 145:215-250 (1981). The strand axis is defined as the average of the least-squares lines fit through the midpoints of sequential C_{α} positions of two central β-strands. The sheet plane is defined as the least-squares plane fit through the C_{α} positions of the residues of the sheet. The sheet axis is defined as the vector perpendicular to the sheet plane that passes through the helix center. Ω is the angle between the strand axis and the helix axis after projection onto the sheet plane; θ is the angle between the helix axis and the sheet plane; h is the distance between the helix center and the sheet plane; σ is the rotation angle about the helix axis. The super-secondary structure parameter values for native Gβ1 are Ω=−26.49°, θ=3.20°, h=10.04 Å and σ=0°.

FIGS. 6A, 6B, 6C and 6D depict four supersecondary structure parameters for β/β protein interactions. FIGS. 6A and 6B are relevant to β barrel proteins; FIG. 6C is relevant to β-sheet interactions. FIG. 6A shows only three strands, and depicts R, the barrel radius; α, the tilt of the strands relative to the barrel axis; a, the distance from C^{α} to C^{α} along the strands; and b, the interstrand distance. FIG. 6B shows the twist and coiling angles of the β-sheet, with residues A, B and C from one strand, residues D, E and F in strand 2, and residues G, H and I from strand 3. The circles represent the positions of the residues when projected onto the surface of the barrel. In this case, θ is the mean twist of the β-sheet about an axis perpendicular to the strand direction. T is the mean twist of the β-sheet about an axis parallel to the strand direction. ε is the mean coiling of the β-sheet along the strands. η is the mean coiling of the β-sheet along a line perpendicular to the strands. FIG. 6C depicts two β-sheets, with the chain direction being shown with arrows. FIG. 6D depicts two β-sheets of distance h with angle θ between the average strand vectors. There is also Φ, perpendicular to vectors defining θ.

FIGS. 7A, 7B, 7C and 7D depict four supersecondary structure parameters α/α supersecondary structure parameters for α/α interactions. d is the distance between the helices and θ is the angle between the axes of the helices. σ is defined as the rotation around the helix axis. Ω is the angle between two strand axes after projection onto a plane. In FIGS. 7C and 7D, the dark circle represents a view of the helix from the end.

FIG. 8 illustrates the convergence of HERO compared to DEE (s=2_{mb}) for 37 core residues of a designed leucine rich repeat backbone.

FIGS. 9A and 9B illustrate rotamer notation for dead-end elimination. The thick line represents the protein backbone, the filled circles indicate residue positions, and the thin lines emanating from each residue are side-chain rotamers. FIG. 9A depicts original and Goldstein DEE. FIG. 9B depicts simple split DEE (s=1).

FIGS. 10A-10D illustrate different dead-end elimination criteria for sample energy profiles. The abscissa represents all possible conformations of the protein and the ordinate describes the net energy contribution produced by interactions with specific rotamers at position i. FIG. 10A depicts original DEE: iᵣ is eliminated by iₜ₁ but not by iₜ₂. FIG. 10B depicts simple Goldstein DEE: iᵣ is eliminated by either iₜ₁ or iₜ₂. FIG. 10C depicts general Goldstein DEE: iᵣ cannot be eliminated by either iₜ₁ or iₜ₂, but can be eliminated by a weighted average of the two. FIG. 10D depicts bottom line DEE: theoretically, iᵣ can be eliminated if the minimum of the iₜ₁ and iₜ₂ profiles always falls below the iᵣ profile.

FIGS. 11A-11D illustrate application of split DEE to sample energy profiles.

FIG. 12 depicts the cost comparisons of different flagging approaches.

FIG. 13. Comparison of bounding and pairs energies during a bound flags iteration of the plastocyanin core calculation of Case 1. The reference energy obtained by a Monte Carlo calculation is shown as a horizontal line. All pairs with a bounding energy above the line may be flagged.

FIGS. 14A-14E depict DEE and HERO convergence results. FIG. 14A illustrates Case 1, full core design of plastocyanin. FIG. 14B illustrates Case 2, full core design of novel repeating backbone. FIG. 14C illustrates Case 3, full core design of the variable domains of the light and heavy chains of a catalytic antibody. FIG. 14D illustrates Case 4, full core and boundary design of the β1 domain of Protein G. FIG. 14E illustrates Case 5, full surface design of the β1 domain of Protein G.

FIG. 15 illustrates convergence comparison for HERO on a full sequence design of the P1 domain of Protein G using the experimentally validated “standard” potential function and rotamer library, a modified potential function with the standard rotamer library and the standard potential function with a reduced rotamer library.

### DETAILED DESCRIPTION OF THE INVENTION

The present invention is directed to the quantitative design and optimization of amino acid sequences, using an “inverse protein folding” approach, which seeks the optimal sequence for a desired structure. Inverse folding is similar to protein design, which seeks to find a sequence or set of sequences that will fold into a desired structure. These approaches can be contrasted with a “protein folding” approach which attempts to predict a structure taken by a given sequence.

The general preferred approach of the present invention is as follows, although alternate embodiments are discussed below. A known protein structure is used as the starting point. The residues to be optimized are then identified, which may be the entire sequence or subset(s) thereof. The side chains of any positions to be varied are then removed. The resulting structure consisting of the protein backbone and the remaining sidechains is called the template. Each variable residue position is then preferably classified as a core residue, a surface residue, or a boundary residue; each classification defines a subset of possible amino acid residues for the position (for example, core residues generally will be selected from the set of hydrophobic residues, surface residues generally will be selected from the hydrophilic residues, and boundary residues may be either). Each amino acid can be represented by a discrete set of all allowed conformers of each side chain, called rotamers. Thus, to arrive at an optimal sequence for a backbone, all possible sequences of rotamers must be screened, where each backbone position can be occupied either by each amino acid in all its possible rotameric states, or a subset of amino acids, and thus a subset of rotamers.

Two sets of interactions are then calculated for each rotamer at every position: the interaction of the rotamer side chain with all or part of the backbone (the “singles” energy, also called the rotamer/template or rotamer/backbone energy), and the interaction of the rotamer side chain with all other possible rotamers at every other position or a subset of the other positions (the “doubles” energy, also called the rotamer/rotamer energy). The energy of each of these interactions is calculated through the use of a variety of scoring functions, which include the energy of van der Waal's forces, the energy of hydrogen bonding, the energy of secondary structure propensity, the energy of surface area solvation and the electrostatics. Thus, the total energy of each rotamer interaction, both with the backbone and other rotamers, is calculated, and stored in a matrix form. Thus, a sample matrix is generated for the singles calculation, and for the doubles calculations.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, either a “Dead End Elimination” (DEE) calculation or a “Hybrid Exact Rotamer Optimization” (HERO) calculation, or both, are performed. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably. DEE can be rerun comparing pairs of rotamers, or combinations of rotamers, which will eventually result in the determination of a single sequence which represents the global optimum energy. Alternatively, a HERO calculation can be done.

In a preferred embodiment, a Hybrid Exact Rotamer Optimization (HERO) calculation is done to find the single sequence which represents the global optimum energy. Thus, the computational processing includes one or more HERO computational steps as outlined below.

Accordingly, the present invention provides HERO, an exact algorithm for determining the global minimum energy conformation (GMEC) of a set of discrete side chain rotamers anchored to fixed protein backbone coordinates. For large design problems, HERO converges significantly faster than existing exact algorithms, and provides solutions for design cases that were previously intractable by any known exact search method.

HERO employs both dominance criteria and bounding criteria to eliminate single rotamers, and flag pairs of rotamers that are incompatible with the GMEC conformation. The bounding criteria require a reliable cutoff energy from a valid conformation. This is obtained during the calculation using parallel Monte Carlo searches from the current state of the HERO conformational ensemble.

The dominance criteria were originally developed for use in the Dead-End Elimination (DEE) algorithm and the bounding criteria were originally developed for use in Branch and Terminate (B & T) (Gordon, D B., and Mayo, S L., (1999) Structure, 7:1089-1097, hereby incorporated by reference in its entirety). While the method employs a stochastic Monte Carlo search to inform the bounding criteria, HERO remains an exact search method. The novelty of HERO is the simultaneous incorporation of three completely different search paradigms into a single compatible exact search algorithm.

In a preferred embodiment, HERO comprises three search paradigms into a single exact search algorithm. The three search paradigms comprise dominance criteria, bounding criteria, and a stochastic search paradigm, wherein all three search paradigms are run simultaneously. These three search paradigms are facilitated by the process of unification, which is described below with particular reference to “super residues”.

Dominance criteria comprise Dead-end elimination algorithms. By “Dead-end elimination algorithms” herein is meant the original DEE theorem or a variation thereof (as outlined below). Thus, DEE algorithms which may be used include: original DEE, simple Goldstein DEE (T=1; Equation 36), general Goldstein DEE (T>1; Equation 37), Goldstein DEE doubles (described below), magic bullet Goldsteins doubles (described below); bottom line DEE, simple split DEE (s=1; Equation 38); split singles DEE (s=2; Equation 44); split singles DEE (s>1; Equation 39); magic bullet splitting (also referred to herein as magic bullet metric; Equation 40); and, split flags (Equation 45). The dominance criteria may be used alone or in combination.

Bounding criteria comprise singles bounding criteria (Equation 42) and doubles bounding criteria (Equation 43).

Preferably, the stochastic search paradigm comprises a Monte Carlo search.

Accordingly, the present invention describes a new version of singles DEE that effectively splits the conformational space into partitions. A rotamer at a given position can then be eliminated if, within zoo each of the partitions, at least one of the other candidate rotamers at that location always produces lower pairwise interaction energies. A hierarchy of splittings is defined, the simplest of which remains O(n³p²), while substantially increasing the number of rotamers that are identified as dead-ending. Here p is the number of residue positions and n is the average number of rotamers per position.

Using a potential function described in terms of pairwise interactions, the total energy of a protein can be evaluated with:

$${E}_{total} = {E}_{template} + {\sum }_{i} E ( {i}_{r} ) + {\sum }_{i} {\sum }_{j , j < i} E ( {i}_{r} {j}_{u} ) \qquad \text{Equation 34}$$

Here, Eₜₑₘₚₗₐₜₑ represents the self-energy of the back-bone and E(iᵣ) represents the energy of rotamer r at position i, including both the self-energy and the interaction energy with the backbone. The term E(iᵣ,jᵤ) represents the interaction energy between rotamers r and u at positions i and j, respectively. It is precisely the calculation of this total energy for each possible conformation that DEE seeks to avoid.

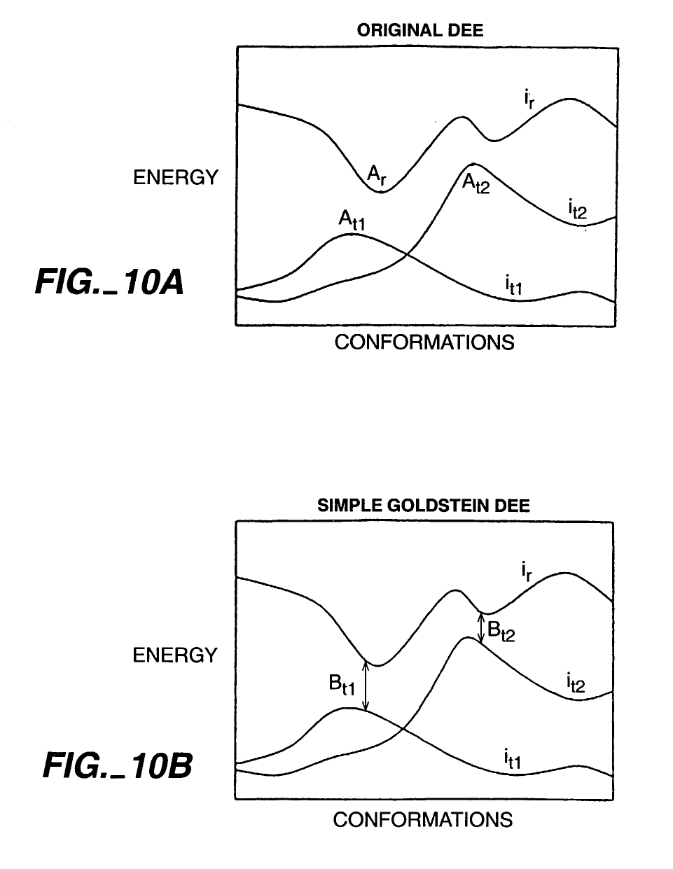

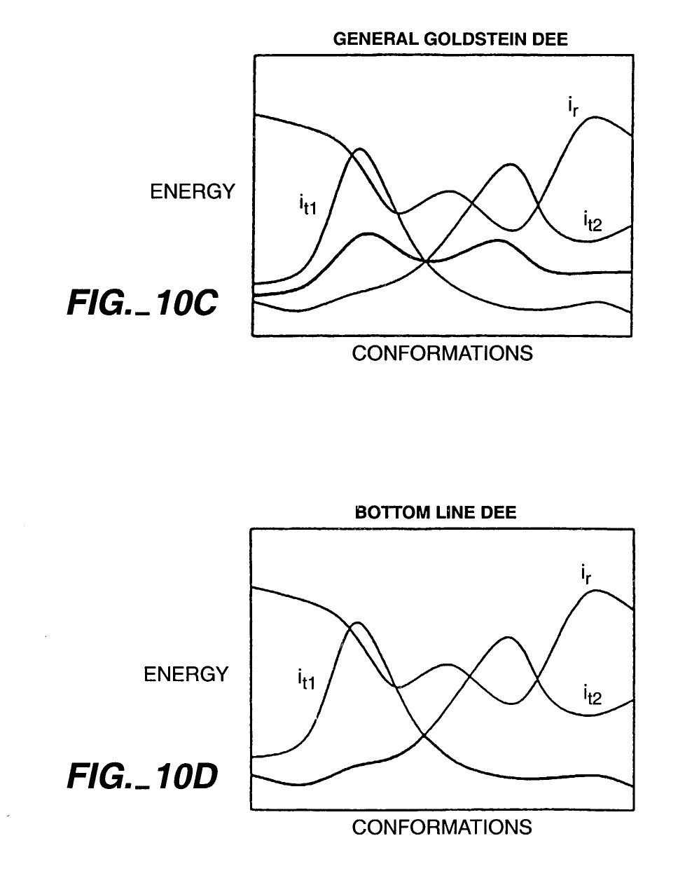

The original dead-end elimination criterion states that a rotamer, iᵣ, can be eliminated if an alternative rotamer, it, at the same position (see FIG. 10A) satisfies:

$$E ( {i}_{r} ) + {\sum }_{j , j \neq i} {min}_{u} E ( {i}_{r} {j}_{u} ) > E ( {i}_{t} ) + {\sum }_{j , j \neq i} {max}_{u} E ( {i}_{t} , {j}_{u} ) \qquad \text{Equation 35}$$

This condition implies that iᵣ can be eliminated if the net energy contribution resulting from its best case pairwise interactions with rotamers at all other positions (spanned by jᵤ) is still worse than that produced by the worst case pairwise interactions of some other candidate rotamer, iₜ, at the same position. To help in visualizing the significance of this criterion, FIG. 10A depicts some relevant energy landscapes. Here, the abscissa represents all possible conformations of the protein and the ordinate describes the net energy contribution produced by interactions with specific rotamers at position i. It is important to note that these energy profiles are not actually continuous and are instead composed of discrete points displayed in some arbitrary ordering of conformational space. The left-hand side of eq. (35) identifies the energy corresponding to the best case conformation Aᵣ for rotamer iᵣ, and the right-hand side identifies the energies for worst case conformations Aₜ₁, and Aₜ₂ for candidate rotamers iₜ₁ and iₜ₂,respectively. Hence, in the present scenario, rotamer iᵣ could be eliminated by rotamer iₜ₁ but not by iₜ₂.

Goldstein (Goldstein, R F. (1994) J. Prot. Eng., 66:1335) improved on this idea using the more powerful criterion shown in Equation 36 (“Simple Goldstein DEE (T=1)):

$$E ( {i}_{r} ) - E ( {i}_{t} ) + {\sum }_{j , j \neq i} \{ {min}_{u} [ E ( {i}_{r} {j}_{u} ) - E ( {i}_{t} {j}_{u} ) ] \} > 0 \qquad \text{Equation 36}$$

which states that rotamer iᵣ can be eliminated if the contribution to the total energy is always reduced by using an alternative rotamer, iₜ. In FIG. 10B, the criterion measures the minimum difference between the profile for iᵣ and the profiles using other candidate rotamers. In the present example, rotamers iₜ₁ and itₜ₂ are both able to eliminate iᵣ, because the differences at the points of closest approach (Bₜ₁ and Bₜ₂, respectively) are positive in both cases. This increased elimination power is a tremendous advantage in reducing the combinatorial size of the problem prior to resorting to doubles calculations. However, the criterion is still unable to eliminate rotamer iᵣ if all of the candidate iₜ rotamers have energy profiles that cross the iᵣ profile for at least one conformation.

To attempt to cope with this more challenging scenario, Goldstein proposed a more general criterion (T>1):

$$E ( {i}_{r} ) - {\sum }_{t = 1 , T} {C}_{t} E ( {i}_{t} ) + {\sum }_{j , j \neq i} \{ {min}_{u} [ E ( {i}_{r} {j}_{u} ) - {\sum }_{t = 1 , T} {C}_{t} E ( {i}_{t} {j}_{u} ) ] \} > 0$$

which uses a weighted average of T candidate iₜ rotamers to attempt to eliminate iᵣ. If some average of candidates is always better than the energy produced by iᵣ, it then follows that there is always at least one candidate it that provides a better alternative to iᵣ, regardless of which conformation turns out to be the GMEC. FIG. 10C illustrates a case for which iᵣ can be eliminated by the average of iₜ₁ and iₜ₂ with constants Cₜ₁=Cₜ₂=½. One complication with this approach is that the choice of constants is not obvious, although Lasters et al. (Lasters, I., et al. (1997), J. Prot. Chem., 16:449-452) demonstrated that linear programming can be used to efficiently select suitable weights Cₜ. However, even if the optimal weights can be identified, this criterion is still more conservative than the most general idea underlying DEE. In a case in which none of the candidate iₜ rotamers have lower energies than iᵣ for all conformations (and hence cannot eliminate iᵣ by themselves), then they will necessarily be raising the average of the hybrid somewhere in conformation space.

Theoretically, it is unnecessary for the same iₜ rotamer to eliminate iᵣ for all regions of conformational space. Instead rotamer iᵣ can be eliminated if at least one candidate rotamer produces a lower energy for each possible conformation. As illustrated in FIG. 10D, rotamer iᵣ could thus be eliminated if the “bottom line” (Desmet, J., et al. In Altman, RB, et al (eds) Pacific Symposium on Biocomputing 1997, world Scientific: Singapore, 1997, p.122) taken as the minimum energy of all the other possible alternative it candidates was always less than that produced using iᵣ. Although the bottom line criterion is theoretically the most powerful DEE elimination criterion, it is not apparent how to implement the approach.

Alternatively, it is possible to split conformational space into partitions, within which each of the candidate iₜ rotamers can be compared singly with iᵣ. If at least one it rotamer produces a lower energy for each partition, then iᵣ can be eliminated even if no single iₜ satisfies the simple Goldstein criterion for all partitions. Hence, for suitably defined partitions, it would be possible for rotamers iₜ₁ and iₜ₂ of FIG. 10D to jointly eliminate rotamer it, even though neither of them could accomplish the feat alone.

The partitioning approach adopted herein is to split the conformational space into O(n) equally sized partitions using the rotamers at some position k≠i.

In a preferred embodiment, simple split DEE (s=1) is used to split conformation space into partitions. The criterion for simple split DEE (s=1) is shown in Equation 38:

$$E ( {i}_{r} ) - E ( {i}_{t} ) + {\sum }_{j , j \neq k \neq i} \{ {min}_{u} [ E ( {i}_{r} {j}_{u} ) - E ( {i}_{t} {j}_{u} ) ] \} + [ E ( {i}_{r} {k}_{v} ) - E ( {i}_{t} {k}_{v} ) ] > 0 \qquad \text{Equation 38}$$

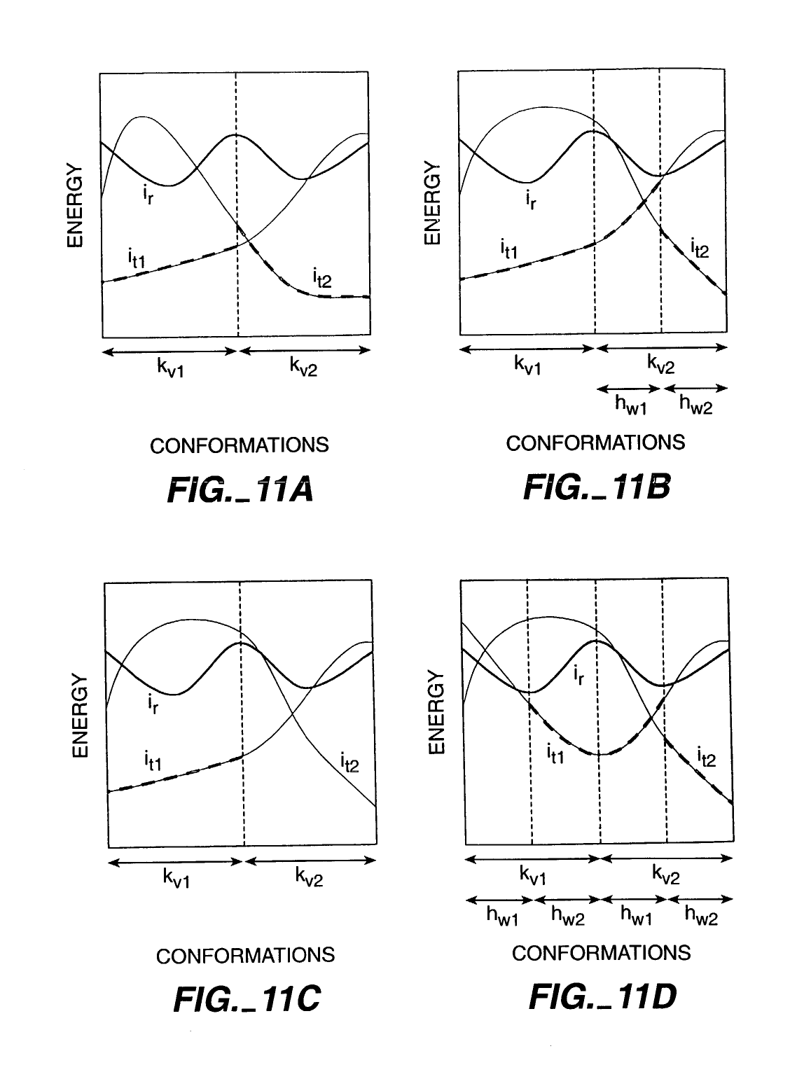

stating that iᵣ can be eliminated if, for each splitting rotamer v, at some splitting position k≠i, there exists an iₜ rotamer that yields a lower net energy contribution for all conformations within that partition. Domination in partition kᵥ is automatic if (iᵣ,kᵥ) is a flagged pair. The splitting position k is thus removed from the summation in simple Goldstein DEE so that the relative merits of iᵣ and the various iₜ candidates can be evaluated for each of the splitting rotamers kᵥ corresponding to individual partitions of the conformational space. The split DEE criterion is illustrated in FIG. 11A. FIG. 10E illustrates a case in which iᵣ is successfully eliminated by splitting the conformation space into two partitions corresponding to the splitting rotamers kᵥ₁ and kᵥ₂.

In a preferred embodiment, the number of splitting positions is increased. Increasing the number of splitting positions increase both the elimination power and the computational complexity. For example, for two splitting positions (s=2), there are O(n²) partitions and i, may be eliminated if, for each pair of splitting rotamers kᵥ and h_{w} at splitting positions k≠h≠i, there exists an iₜ rotamer that dominates iᵣ in that partition:

$$E ( {i}_{r} ) - E ( {i}_{t} ) + {\sum }_{j , j \neq i \neq h \neq k} \{ {min}_{{u}_{( {i}_{r} , {j}_{u} ) ∈ F}} [ E ( {i}_{r} , {j}_{u} ) - E ( {i}_{t} , {j}_{u} ) ] \} + [ E ( {i}_{r} , {k}_{v} ) - E ( {i}_{t} , {k}_{v} ) ] + [ E ( {i}_{r} , {h}_{w} ) - E ( {i}_{t} , {h}_{w} ) ] > 0 \qquad \text{Equation 44}$$

Domination follows automatically if either (iᵣ,kᵥ) or (iᵣ,h_{w}) is a flagged pair. The application of this criterion is illustrated in FIG. 11B, where the rotamers at the second splitting position (h_{w}) effectively create sub-partitions of those created by the first position (kᵥ). The computational complexity for (s=2) split DEE is O(n⁴p³).

In a preferred embodiment, “split singles” DEE (s>1) is used to expand a larger fraction of the combinatorial space by using s splitting locations simultaneously, corresponding to O(n^{s}) partitions. Split singles DEE (s>1) criterion is defined: eliminate iᵣ if for all unique combinations of v at some:

k₁ . . . kₛ≠i

there exists an iₜ such that Equation 39 holds:

$$E ( {i}_{r} ) - E ( {i}_{t} ) + {\sum }_{j , j \neq {k}_{i} ks \neq i} {min}_{u} [ E ( {i}_{r} {j}_{u} ) - E ( {i}_{t} {j}_{u} ) ] + {\sum }_{k = {k}_{1} ks} [ E ( {i}_{r} {k}_{v} ) - E ( {i}_{t} {k}_{v} ) ] > 0 \qquad \text{Equation 39}$$

This criterion may be applied for any s in the range 2, 3, 4, . . . , p−1, where p is the total number of residue positions in the design.

As the number of splitting positions and partitions increase, so does the cost per iteration.

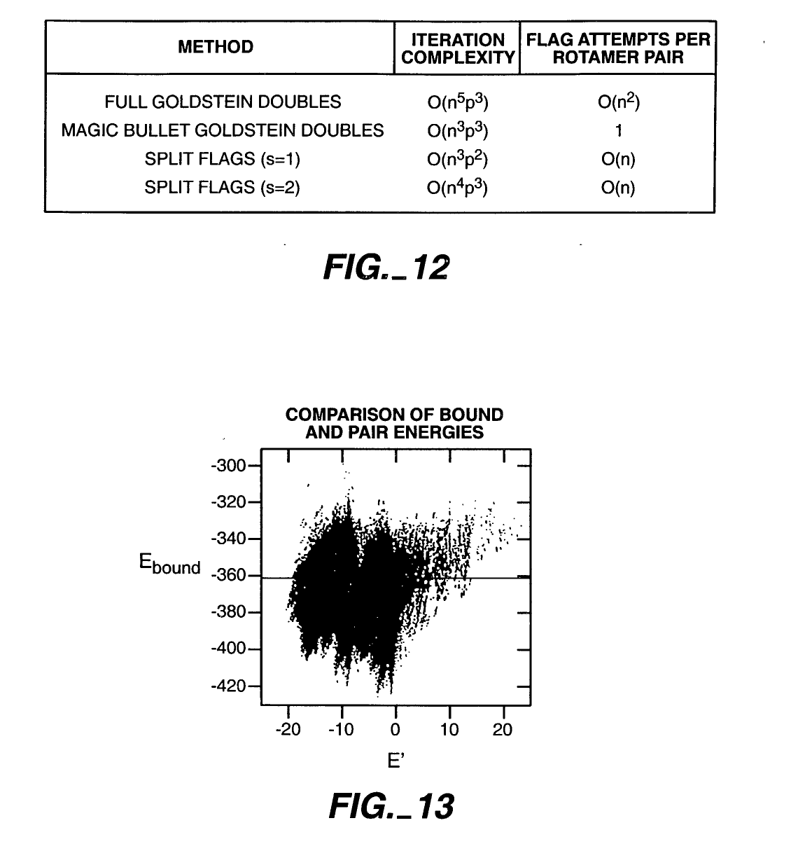

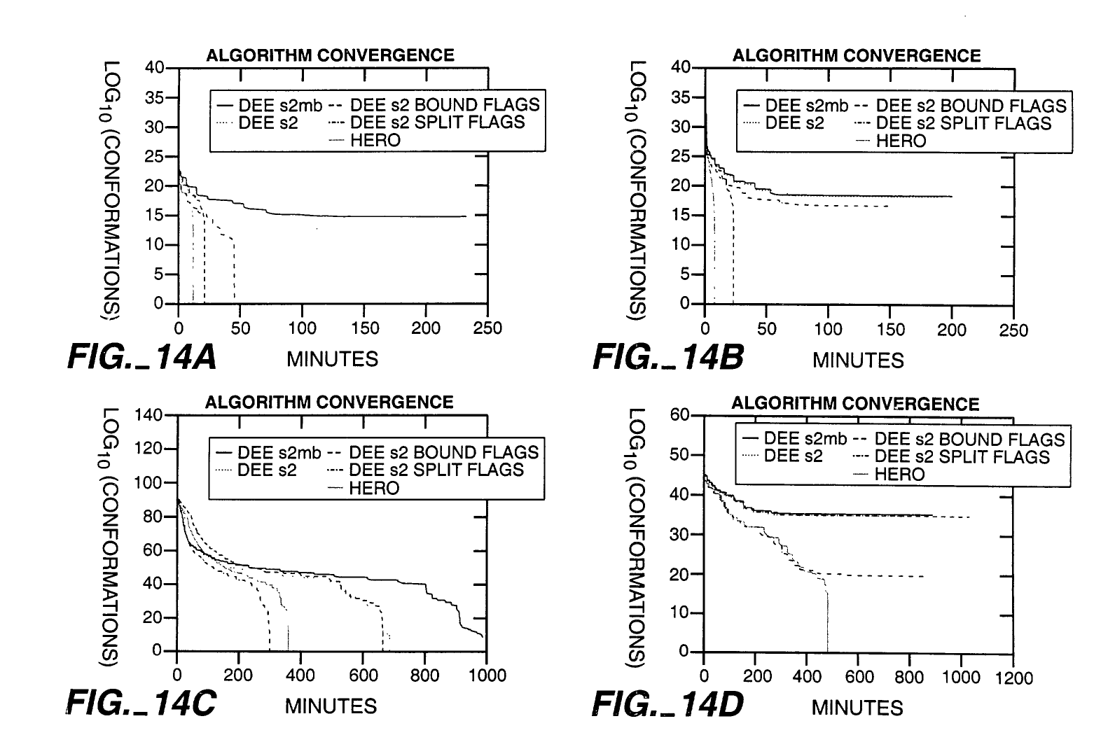

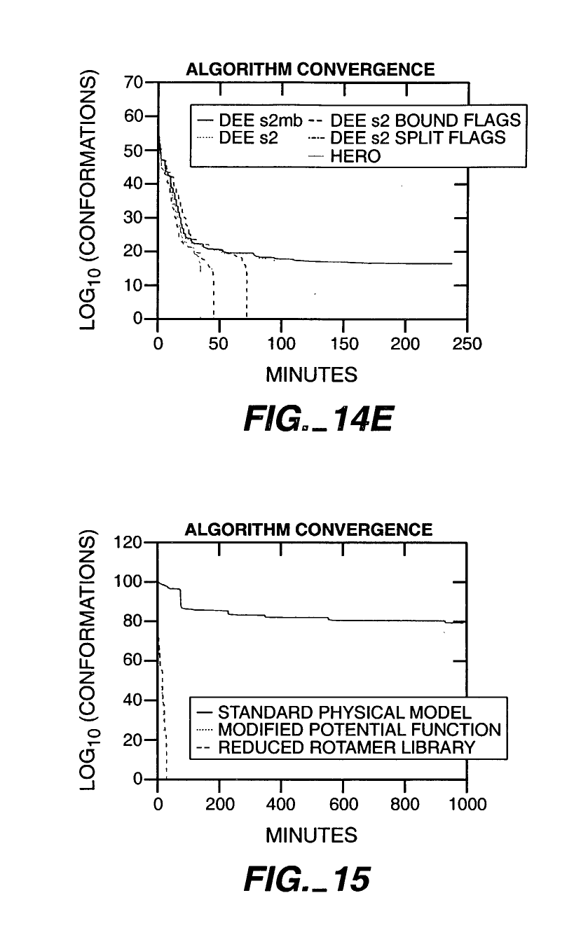

Although split DEE criteria may be extended to flag pairs of rotamers exactly as for the Goldstein doubles criterion (as described below), flags (i.e., split flags) also can be generated during split singles calculations with no increase in computational complexity (See FIG. 12). This concept is illustrated in FIGS. 11A-11D. In FIGS. 11A-11D, the abscissa represents all possible conformations of the protein and the ordinate represents the net energy contributions produced by interactions with specific rotamers at position iᵣ.

FIG. 11A depicts split DEE elimination (s=1). iᵣ is dominated by iₜ₁ and iₜ₂ in the partitions corresponding to splitting rotamers kᵥ₁ and kᵥ₂, respectively. Hence, iᵣ may be eliminated even though it is not dominated by any single rotamer for all of conformational space. FIG. 11C illustrates a scenario in which iᵣ cannot be eliminated by split DEE (s=1) because there are some partitions in which no iₜ rotamer dominates iᵣ. In this case, it is still possible to identify dead-ending pairs during the process of discovering this negative result. In those partitions kᵥ where iᵣ is dominated by some iₜ, the rotamer pairs (iᵣ,kᵥ) may be flagged as dead-ending. This concept is illustrated in FIG. 11C. The comparisons that are made in an effort to identify flags remain a subset of those made for a full Goldstein doubles calculation. O(n) dominance comparisons are made in attempting to flag each rotamer pair. The complexity of split DEE is unaffected by this modification, remaining O(n³p²) for (s=1.)

FIG. 11B depicts split elimination (s=2). As neither iₜ₁ or iₜ₂ dominates iᵣ in partition kᵥ₂, a second splitting position is used to create sub-partitions h_{w1} and h_{w2}, where iᵣ is dominated by iₜ₁ and iₜ₂, respectively. Thus, iᵣ may be eliminated using two splitting positions. FIG. 11 depicts split flagging (s=1). iᵣ is not dominated in partition kᵥ₂ so elimination is not possible with only one splitting position. However, iᵣ is dominated by iₜ₁ in partition kᵥ₁ so that pair (iᵣ,kᵥ₁) may be flagged. FIG. 11D depicts split flagging (s=2). iᵣ is no longer dominated for all of conformational space so it cannot be eliminated with only two splitting positions. However, iᵣ is dominated for partition kᵥ₂ by iₜ₁ and iₜ₂ in sub-partitions h_{w1} and h_{w2}, respectively. Hence, the pair (iᵣ,kᵥ₂) may be flagged. Likewise, the pair (iᵣ,h_{w2}) may be flagged, as becomes more readily apparent if the hierarchy of the splitting positions k and h is reversed.

In a preferred embodiment, split singles DEE with split flags are generated with arbitrary numbers of splitting positions. This concept is illustrated for split DEE (s=2) in FIG. 11D. In split flags, the number of flagging comparisons remains O(n) per rotamer pair but the iteration complexity increases to O(n⁴p³). For a given maximum number of splitting positions, the attempt to eliminate a rotamer iᵣ fails as soon as a sub-partition at the lowest level is encountered in which iᵣ is not dominated by some competitor. See also Looger, L. L., and Hellinga, H. W. (2001) J. Mol. Biol., 307:429-445 for the special case of s=1.

In a preferred embodiment, split singles DEE flags with split flags (s=1) is used until no further eliminations are possible.

In a preferred embodiment, split singles DEE with split flags (s≧2) is run once for each rotamer with or without magic bullet metric (equation 40).

The singles criteria described above form the heart of the DEE approach. However, there are a number of refinements based on this foundation that substantially increase the performance of the algorithm. The most important of these is the use of doubles criteria to flag dead ending pairs for more efficient elimination using the singles criteria . Other refinements include a magic bullet doubles version of Goldstein criterion outlined below (see also Gordon, D B., and Mayo, S L. (1998) J. Comput. Chem., 19:1505; incorporated herein in its entirety). Magic bullet splitting may also be used to improve the performance of split singles DEE.

When considering the use of split singles DEE, it is advantageous to keep the complexity well below O(n⁵p³), so as to keep the cost of singles elimination a small fraction of the more expensive doubles process. The complexity estimate for split singles (Equation 39) reveals that the cost grows rapidly as s is increased. For s=1, the cost is O(n³p²), which is identical to the estimate for simple Goldstein singles. To reduce the cost for s>1, it is desirable to use only the one “magic bullet” splitting k₁ . . . kₛ that appears most likely to eliminate rotamer iᵣ; in this case, the cost of magic bullet split DEE reduces to O(n^{2+s}p)

In a preferred embodiment, magic bullet split singles DEE is used. Intuitively, the best splitting locations for magic bullet split single DEE should be those that have strong interactions with the rotamers at i. Accordingly, the objective is to find splitting positions k₁ . . . kₛ, such that the candidate iₜ rotamers have widely varying interaction energies with rotamers at these positions k₁ . . . kₛ. Assuming no single iₜ rotamer is able to eliminate iᵣ, the splitting will then enable other candidate iₜ rotamers to contribute to the elimination of iᵣ. For each iᵣ, the following “magic bullet metric” can be used to rank the positions k₁ . . . kₛ with which the iₜ rotamers have the strongest adverse interactions:

$${min}_{k} {min}_{t} {min}_{v} [ E ( {i}_{r} {k}_{v} ) - E ( {i}_{t} {k}_{v} ) ] \qquad \text{Equation 40}$$

For s=2 magic bullet splitting (s=2_{mb}), rotamers at the top two ranked positions are then used to split the conformational space, corresponding to a complexity of O(n⁴p). Using magic bullet splitting pairs is thus only a factor of O(n/p) more expensive than using s=1 splitting performed at all positions. For s=3, magic bullet splitting triplets (s=3_{mb})with a cost bound of O(n⁵p) are also less expensive than Goldstein doubles.

In addition to the DEE dominance criteria described above, HERO also uses bounding criteria to eliminate rotamers and flag rotamer pairs that are incompatible with the GMEC. The bounding criteria for single rotamers and pairs of rotamers are special cases of the criteria used for B & T. The bounding criteria are summarized in Equations 41-43, using the following notation:

$${E}_{pair} ( {i}_{r} , {j}_{u} ) = \frac{E ( {i}_{r} ) + E ( {j}_{u} )}{2 p - 2} + \frac{E ( {i}_{r} , {j}_{u} )}{2} , \qquad \text{Equation 41}$$

where p is the number of positions in the design. The energy bound for a single rotamer, referred to herein as “singles bounding criteria” is shown in Equation 42:

$${E}_{bound} ( {i}_{r} ) = {\sum }_{k \neq i} {min}_{t} \{ 2 {E}_{pair} ( {i}_{r} , {k}_{t} ) + {\sum }_{j \neq k \neq i} {min}_{u} [ {E}_{pair} ( {j}_{u} {k}_{t} ) ] \} , \qquad \text{Equation 42}$$

and iᵣ can be eliminated if E_{bound}(iᵣ)>E_{low}, where E_{low} is the lowest known energy of a valid conformation determined by the Monte Carlo searches. The energy bound for a pair of rotamers, referred to herein as “doubles bounding criteria” is shown in Equation 43:

$${E}_{bound} ( {i}_{r} , {j}_{u} ) = 2 {E}_{pair} ( {i}_{r} , {j}_{u} ) + {\sum }_{k \neq i \neq j} {min}_{t} \{ 2 {E}_{pair} ( {i}_{r} , {k}_{t} ) + 2 {E}_{pair} ( {j}_{u} {k}_{t} ) + {\sum }_{m \neq k \neq j \neq i} {min}_{v} [ {E}_{pair} ( {m}_{v} , {k}_{t} ) ] \} , \qquad \text{Equation 43}$$

and pair (iᵣ, jᵤ) can be flagged if E_{bound}(iᵣ, jᵤ)>E_{low}.

HERO is parallelized to run across any number of processors. If HERO is run on N processors, then N independent Monte Carlo searches are performed during the stochastic search phase of the algorithm.

Regardless of the DEE or bounding criterion used, it is generally beneficial to apply the condition iteratively, as previous eliminations often facilitate the elimination of further rotamers. Eventually, no further rotamers will be eliminated at any position by additional rounds of DEE or bounding. At this point, it is necessary to resort to a doubles calculation to flag dead-ending pairs, which can then be used to increase the effectiveness of the singles elimination criterion. “Dead Ending Pairs” are pairs of rotamers that have been identified as being incompatible with GMEC. After further applications of a singles elimination criterion, it again becomes impossible to eliminate further rotamers. At this point, the rotamers at two positions are “unified” to form a superresidue that is treated as a single position for the remainder of the calculation. This process permanently expands a fraction of the combinatorial space and sets off a new cascade of singles eliminations. Furthermore, the two unified positions are chosen to be those with the highest fraction of dead-ending pairs. These pairs become dead-ending super-rotamers of the new superresidue and can thus be eliminated at the time of unification.

In a preferred embodiment, HERO is used as the analyzing step to generate a set of optimized protein sequences. By “HERO” herein is meant an algorithm used to identify a single rotamer at each position that belongs to the global minimum energy conformation (GMEC).

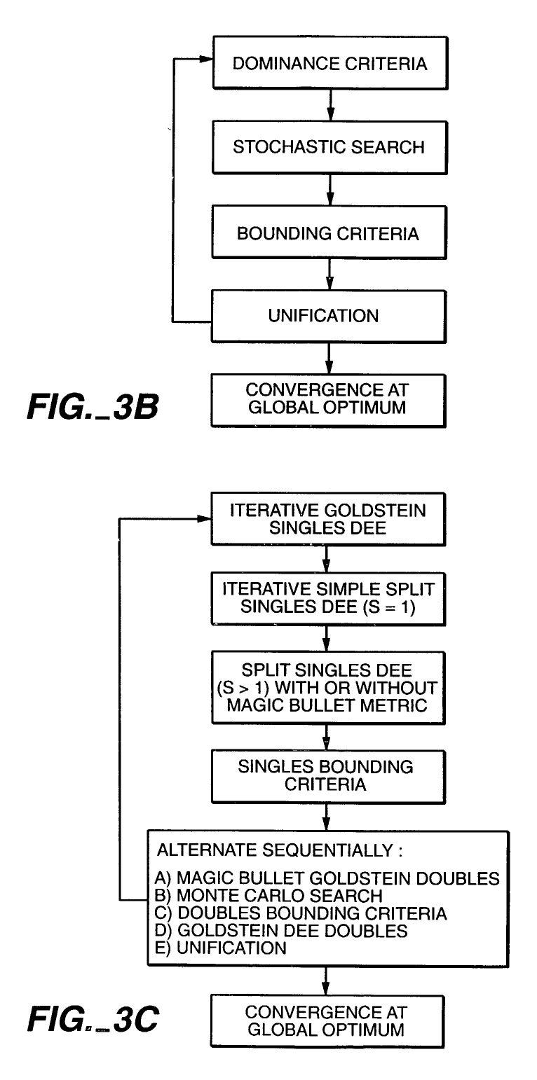

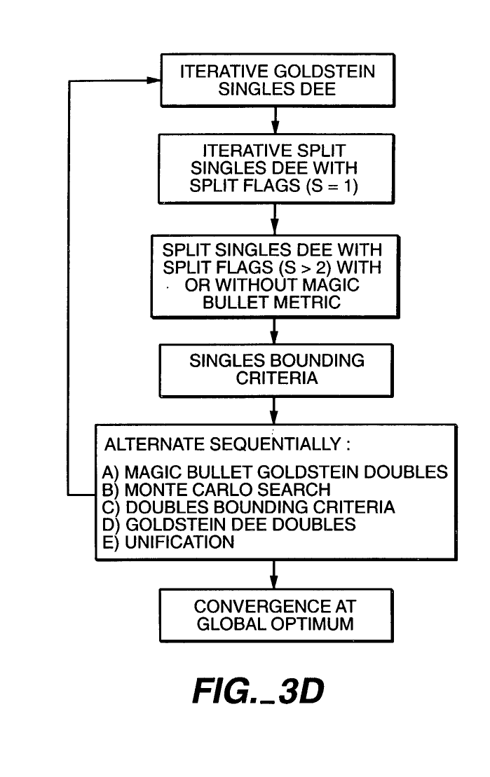

In a preferred embodiment, HERO is run using split singles DEE. In this embodiment, HERO includes the following steps: 1) Goldstein singles DEE (T=1), repeated until no further eliminations can be made; 2) simple split DEE ( s=1), repeated until no further eliminations can be made; 3) split singles sang DEE (s>1) with or without magic bullet metric, once for each rotamer; 4) application of singles bounding criteria to eliminate rotamers whose bound energy E_{bound} is greater than E_{low}, the lowest known energy of a valid conformation obtained from the Monte Carlo searches; 5) alternating sequentially between one of the following, applying one during each cycle: a) magic bullet Goldstein doubles calculation to flag dead ending pairs; b) Monte Carlo search to find a low energy of a valid conformation E_{low}, c) applying doubles bounding criteria to flag pairs whose bounding energy E_{bound} is greater than E_{low}; d) Goldstein DEE doubles calculation to flag dead ending pairs; and, e) unification to identify super residues (see FIG. 3B); and 6) repeating steps 1-5 until the GMEC is found.

In a preferred embodiment, HERO is run using split singles DEE with split flags. In this embodiment, HERO includes the following steps: 1) Goldstein singles DEE, repeated until no further eliminations can be made; 2) split singles DEE with split flags (s=1), repeated until no further eliminations can be made; 3) split singles DEE with split flags (s≧2) once for each rotamer, with or without magic bullet metric; 4) application of singles bounding criteria to eliminate rotamers whose bound energy E_{bound} is greater than E_{low}, the lowest known energy of a valid conformation obtained from the Monte Carlo searches; 5) alternating sequentially between one of the following, applying one during each cycle: a) magic bullet Goldstein doubles calculation to flag dead ending pairs; b) Monte Carlo search to find a low energy of a valid conformation E_{low}, c) applying doubles bounding criteria to flag pairs whose bounding energy E_{bound} is greater than E_{low}; d) Goldstein DEE doubles calculation to flag dead ending pairs; and, e) unification to identify super residues (see FIG. 3B); and 6) repeating steps 1-5 until the GMEC is found.

Once the global solution has been found, a Monte Carlo search may be done to generate a rank-ordered list of sequences in the neighborhood of the DEE or HERO solution. Starting at the DEE or HERO solution, random positions are changed to other rotamers, and the new sequence energy is calculated. If the new sequence meets the criteria for acceptance, it is used as a starting point for another jump. After a predetermined number of jumps, a rank-ordered list of sequences is generated. The results may then be experimentally verified by physically generating one or more of the protein sequences followed by experimental testing. The information obtained from the testing can then be fed back into the analysis, to modify the procedure if necessary.

Thus, the present invention provides a computer-assisted method of designing a protein. The method comprises providing a protein backbone structure with variable residue positions, and then establishing a group of potential rotamers for each of the residue positions. As used herein, the backbone, or template, includes the backbone atoms and any fixed side chains. The interactions between the protein backbone and the potential rotamers, and between pairs of the potential rotamers, are then processed to generate a set of optimized protein sequences, preferably a single global optimum, which then may be used to generate other related sequences.

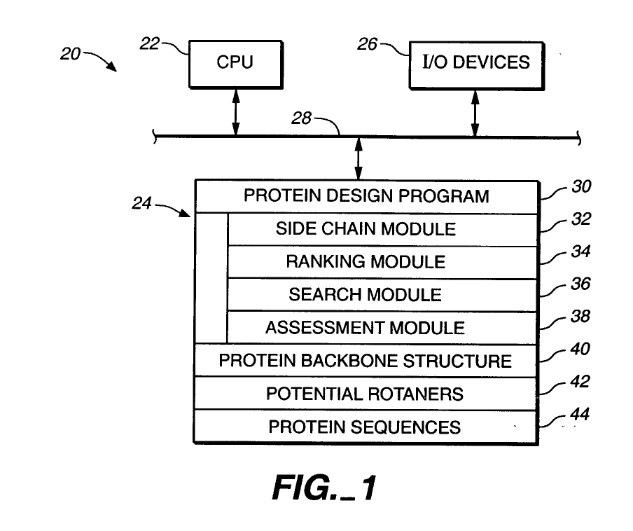

FIG. 1 illustrates an automated protein design apparatus 20 in accordance with an embodiment of the invention. The apparatus 20 includes a central processing unit 22 which communicates with a memory 24 and a set of input/output devices (e.g., keyboard, mouse, monitor, printer, etc.) 26 through a bus 28. The general interaction between a central processing unit 22, a memory 24, input/output devices 26, and a bus 28 is known in the art. The present invention is directed toward the automated protein design program 30 stored in the memory 24.

The automated protein design program 30 may be implemented with a side chain module 32. As discussed in detail below, the side chain module establishes a group of potential rotamers for a selected protein backbone structure. The protein design program 30 may also be implemented with a ranking module 34. As discussed in detail below, the ranking module 34 analyzes the interaction of rotamers with the protein backbone structure to generate optimized protein sequences. The protein design program 30 may also include a search module 36 to execute a search, for example a Monte Carlo search as described below, in relation to the optimized protein sequences. Finally, an assessment module 38 may also be used to assess physical parameters associated with the derived proteins, as discussed further below.

The memory 24 also stores a protein backbone structure 40, which is downloaded by a user through the input/output devices 26. The memory 24 also stores information on potential rotamers derived by the side chain module 32. In addition, the memory 24 stores protein sequences 44 generated by the ranking module 34. The protein sequences 44 may be passed as output to the input/output devices 26.

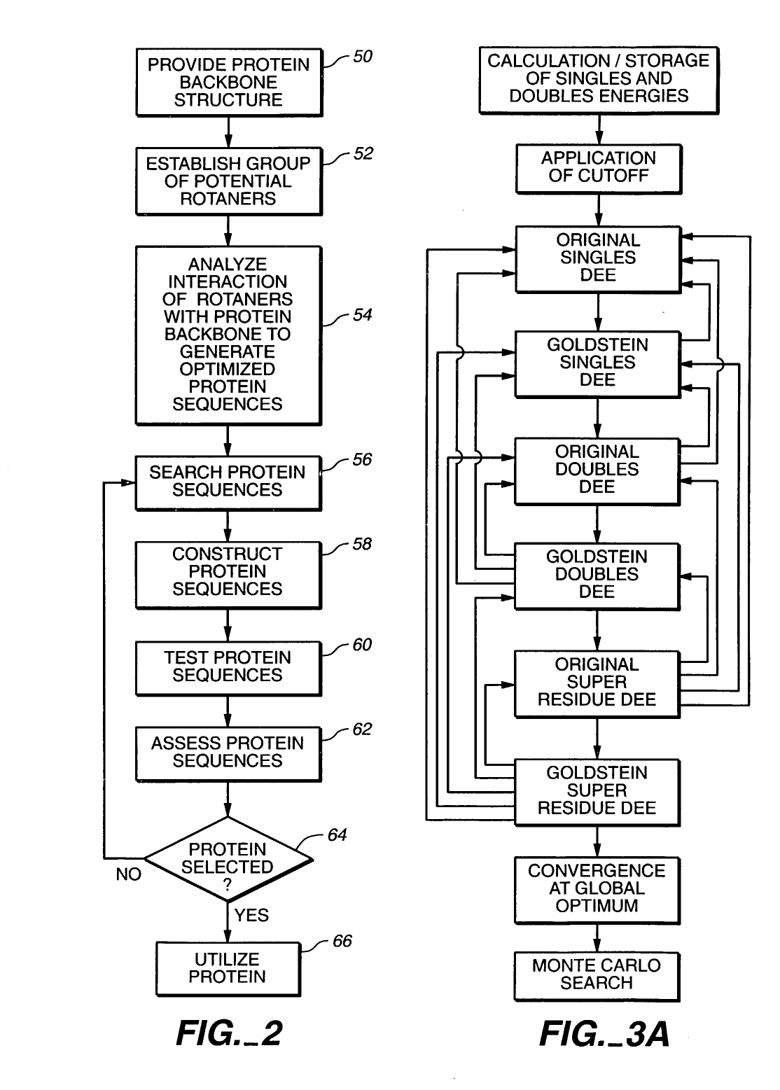

The operation of the automated protein design apparatus 20 is more fully appreciated with reference to FIG. 2. FIG. 2 illustrates processing steps executed in accordance with the method of the invention. As described below, many of the processing steps are executed by the protein design program 30. The first processing step illustrated in FIG. 2 is to provide a protein backbone structure (step 50). As previously indicated, the protein backbone structure is downloaded through the input/output devices 26 using standard techniques.

The protein backbone structure corresponds to a selected protein. By “protein” herein is meant at least two amino acids linked together by a peptide bond. As used herein, protein includes proteins, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992)). The amino acids may either be naturally occuring or non-naturally occuring; as will be appreciated by those in the art, any structure for which a set of rotamers is known or can be generated can be used as an amino acid. The side chains may be in either the (R) or the (S) configuration. In a preferred embodiment, the amino acids are in the (S) or (L) configuration.

The chosen protein may be any protein for which a three dimensional structure is known or can be generated; that is, for which there are three dimensional coordinates for each atom of the protein. Generally this can be determined using X-ray crystallographic techniques, NMR techniques, de novo modelling, homology modelling, etc. In general, if X-ray structures are used, structures at 2 Å resolution or better are preferred, but not required.

The proteins may be from any organism, including prokaryotes and eukaryotes, with enzymes from bacteria, fungi, extremeophiles such as the archebacteria, insects, fish, animals (particularly mammals and particularly human) and birds all possible.

Suitable proteins include, but are not limited to, industrial and pharmaceutical proteins, including ligands, cell surface receptors, antigens, antibodies, cytokines, hormones, and enzymes. Suitable classes of enzymes include, but are not limited to, hydrolases such as proteases, carbohydrases, lipases; isomerases such as racemases, epimerases, tautomerases, or mutases; transferases, kinases, oxidoreductases, and phophatases. Suitable enzymes are listed in the Swiss-Prot enzyme database.

Suitable protein backbones include, but are not limited to, all of those found in the protein data base compiled and serviced by the Brookhaven National Lab.

Specifically included within “protein” are fragments and domains of known proteins, including functional domains such as enzymatic domains, binding domains, etc., and smaller fragments, such as turns, loops, etc. That is, portions of proteins may be used as well. In addition, protein variants, i.e. non-naturally occurring variants, may be used.

Once the protein is chosen, the protein backbone structure is input into the computer. By “protein backbone structure” or grammatical equivalents herein is meant the three dimensional coordinates that define the three dimensional structure of a particular protein. The structures which comprise a protein backbone structure (of a naturally occurring protein) are the nitrogen, the carbonyl carbon, the α-carbon, and the carbonyl oxygen, along with the direction of the vector from the α-carbon to the β-carbon.

The protein backbone structure which is input into the computer can either include the coordinates for both the backbone and the amino acid side chains, or just the backbone, i.e. with the coordinates for the amino acid side chains removed. If the former is done, the side chain atoms of each amino acid of the protein structure may be “stripped” or removed from the structure of a protein, as is known in the art, leaving only the coordinates for the “backbone” atoms (the nitrogen, carbonyl carbon and oxygen, and the α-carbon, and the hydrogens attached to the nitrogen and α-carbon).

In a preferred embodiment, the protein backbone structure is altered prior to the analysis outlined below. In this embodiment, the representation of the starting protein backbone structure is reduced to a description of the spatial arrangement of its secondary structural elements. The relative positions of the secondary structural elements are defined by a set of parameters called supersecondary structure parameters. These parameters are assigned values that can be systematically or randomly varied to alter the arrangement of the secondary structure elements to introduce explicit backbone flexibility. The atomic coordinates of the backbone are then changed to reflect the altered supersecondary structural parameters, and these new coordinates are input into the system for use in the subsequent protein design automation.

Basically, a protein is first parsed into a collection of secondary structural elements which are then abstracted into geometrical objects. For example, as more fully outlined below, an α-helix is represented by its helical axis and geometric center. The relative orientation and distance between these objects are summarized as super-secondary structure parameters. Concerted backbone motion can be introduced by simply modulating a protein's super-secondary structure parameter values. Accordingly, when all or part of the backbone is to be altered, the portion to be altered is classified as belonging to a particular supersecondary structure element, i.e. α/β, α/α or β/β, and then the supersecondary structural elements as outlined below are altered. As will be appreciated by those in the art, these elements need not be covalently linked, i.e. part of the same protein; for example, this can be done to evaluate protein-protein interactions.

As will be appreciated by those in the art, it is possible to alter the backbone of certain positions, while retaining either a particular amino acid (which can be “floated”, as outlined below) or a particular rotamer at the position; alternatively, both the backbone can be moved and the amino acid side chain can be optimized as outlined herein. Similarly, the backbone can be held constant and only the amino acid side chains are optimized. Combinations of any of these at any position may be done. In general, when supersecondary structural parameters are altered, this is done on more than one amino acid, i.e. the backbone atoms of a plurality of amino acids that contribute to the secondary structure are moved.

As will be appreciated by those in the art, there are a wide variety of different supersecondary structure parameters that can be used. Super-secondary structure parameterization has been described for fold classes that include α/α (Crick F H C “The Fourier transform of a coiled-coil.” Acta Crystallogr 6:685-689 (1953a); Crick F H C. “The packing of α-helices.” Acta Crystallogr 6:689-697 (1953b); Chothia et al., Proc Natl Acad Sci USA 78:4146-4150 (1981) “Relative orientation of close-packed β-pleated sheets in proteins”; Chothia et al., J Mol Biol 145:215-250(1981) “Helix to helix packing in proteins”; Chou, et al. “Energetics of the structure of the four-α-helix bundle in proteins.” Proc Natl Acad Sci USA 85:4295-4299 (1988); Murzin A G, Finkelstein A V. “General architecture of the α-helical globule.” J Mol Biol 204:749-769 (1988); Presnell S R, Cohen F E. “Topological distribution of four-α-helix bundles.” Proc Natl Acad Sci USA 86:6592-6596 (1989); Harris et al. “Four helix bundle diversity in globular proteins.” J Mol Biol 236:1356-1368 (1994)); α/β (Chothia et al., “Structure of proteins: packing of α-helices and pleated sheets.” Proc Natl Acad Sci USA 74:4130-4134 (1977); Janin & Chothia, 1980 “Packing of α-helices onto β-pleated sheets and the anatomy of α/β proteins.” J Mol Biol 143:95-128; Cohen et al., 1982, “Analysis and prediction of the packing of α-helices against a β-sheet in the tertiary structure of globular proteins.” J Mol Biol 156:821-862; Chou et al., 1985, “Interactions between an α-helix and β-sheet energetics of α/β packing in proteins.) J Mol Biol 186:591-609); and β/β (Cohen et al., “Analysis and prediction of protein β-sheet structures by a combinatorial approach.” Nature 285:378-382 (1980); Cohen et al., “Analysis of the tertiary structure of protein β-sheet sandwiches.” J Mol Biol 148:253-272 (1981); Chothia & Janin, “Relative orientation of close-packed β-pleated sheets in proteins.” Proc Natl Acad Sci USA 78:4146-4150 (1981); Chothia & Janin, Proc Natl Acad Sci USA 78:3955-3965 (1982) “Orthogonal packing of β-pleated sheets in proteins”; Chou et al., J Mol Biol 188:641-649 (1986) “Interactions between two β-sheets energetics of β/β packing in proteins”; Laster et al., Proc Natl Acad Sci USA 85:3338-3342 (1988)“Structure principles of parallel β-barrels in proteins”; Murzin et al., J Mol Biol 236:1369-1381 (1994a), “Principles determining the structure of β-sheet barrels. I. A theoretical analysis”; Murzin et al. J Mol Biol 236:1382-1400 (1994b) “Principles determining the structure of β-sheet barrels. II. The observed structures”). All of these references are explicitly incorporated by reference herein in their entirety.

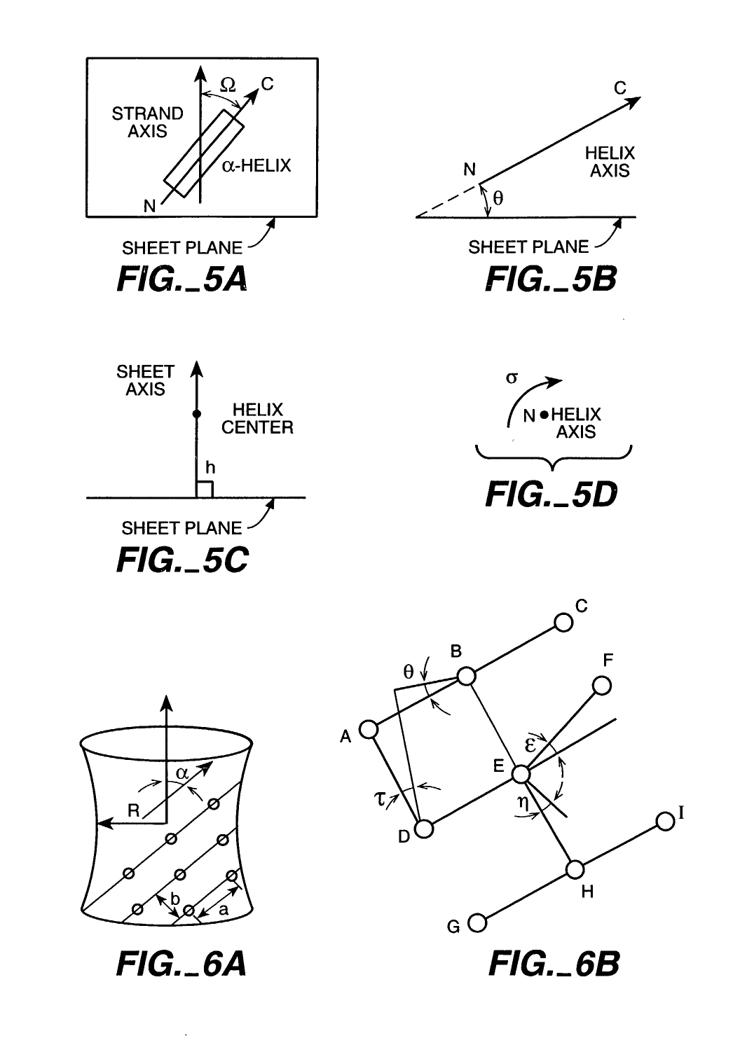

Four different supersecondary structure parameters useful for α/β proteins are shown in FIG. 5. In a preferred embodiment, as for all the supersecondary structure parameters, at least one of these parameter values is altered; other embodiments utilize simultaneous or sequential alteration of two, three or four of these parameter values.

For the α/β protein interactions, the helix center is defined as the average C_{α} position of the residues chosen for backbone movement. The helix axis is defined as the principal moment of the C_{α} atoms of these residues (see Chothia et al., 1981, supra). The strand axis is defined as the average of the least-squares lines fit through the midpoints of sequential C_{α} positions of the two central β-strands.

The sheet plane is defined as the least-squares plane fit through the C_{α} positions of the two central β-strands. The sheet axis is defined as the vector perpendicular to the sheet plane that passes through the helix center. Ω is the angle between the strand axis and the helix axis after projection onto the sheet plane; θ is the angle between the helix axis and the sheet plane; h is the distance between the helix center and the sheet plane; σ is the rotation angle about the helix axis. Backbone alteration requires altering at least one of these parameter values. In a preferred embodiment, the supersecondary structure parameter value Ω is altered by changing the angle degree (either positively or negatively) of up to about 25 degrees, with changes of ±1°, 2.5°, 5°, 7.5°, and 10° being preferred. In a preferred embodiment, the supersecondary structure parameter value θ is altered by changing the angle degree (either positively or negatively) of up to about 25 degrees, with changes of ±1°, 2.5°, 5°, 7.5°, and 10° being particularly preferred. the supersecondary structure parameter value σ is altered by changing the angle degree (either positively or negatively) of up to about 25 degrees, with changes of ±1°, 2.5°, 5°, 7.5°, and 10° being particularly preferred. In a preferred embodiment, the supersecondary structure parameter value h is altered by changes (either positive or negative) of up to about 8 Å, with changes of ±0.25, 0.50, 0.75, 1.00, 1.25 and 1.5 being particularly preferred. However, as will be appreciated by those in the art, as for all the parameter values outlined herein, larger changes can be made, depending on the protein (i.e. how close or far other secondary structure elements are) and whether other parameter values are made; for example, larger changes in Ω can be made if the helix is also moved away from the sheet (i.e. h is increased).

Four different supersecondary structure parameters useful for α/α proteins are shown in FIG. 7. As for α/β parameters, the helix center is defined as the average C_{α} position of the residues in the helix. The helix axis is defined as the principal moment of the C_{α} atoms of the residues in the helix. σ is defined as the rotation around the helix axis. Ω is the angle between two strand axes after projection onto a plane. Thus, d, the distance between the helices, can be altered, generally as outlined above for h. Similarly, θ, σ and Ω can be altered as above.

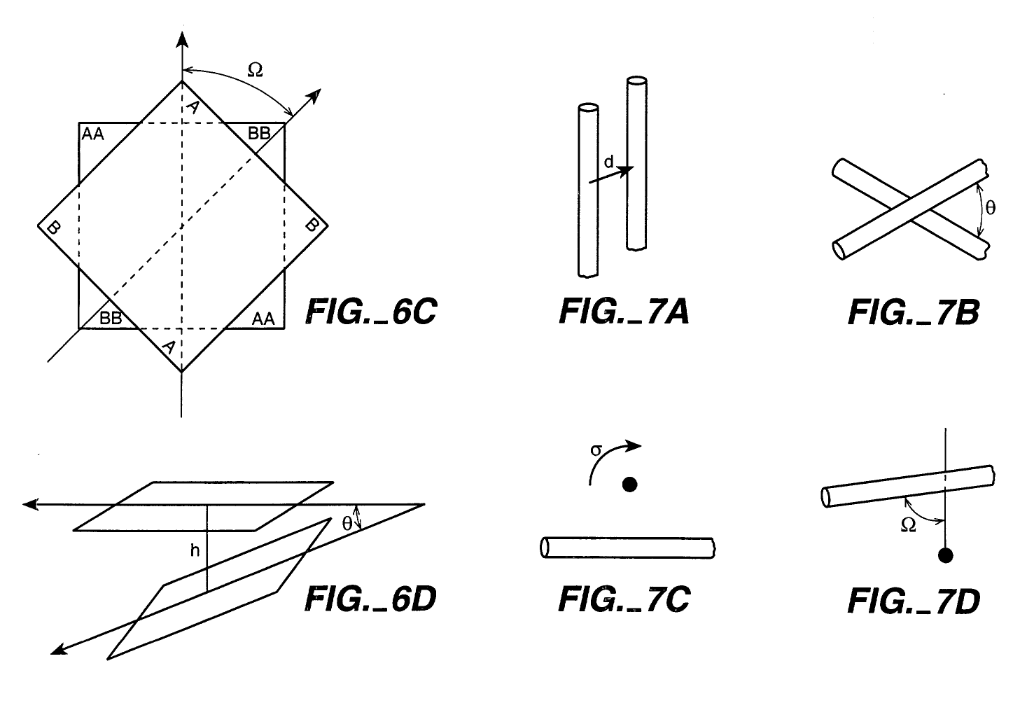

There are a number of different supersecondary structure parameters useful for β/β proteins. β-barrel configurations contain a number of different parameters that can be altered, as shown in FIG. 6. These include: (see FIG. 6A) R, the barrel radius; α the angle of tilt of the strands relative to the barrel axis; and b, the interstrand distance; (see FIG. 6B) θ, the mean twist of the β-sheet about an axis perpendicular to the strand direction; T, the mean twist of the β-sheet about an axis parallel to the strand direction; ε the mean coiling of the β-sheet along the strands; η, the mean coiling of the β-sheet along a line perpendicular to the strands; and (FIG. 6C) Ω is angle between the two β-sheet axes. As for the α/β parameter values, each of these may be altered, either positively or negatively. Generally, changes are made in at least one of these parameter values, by changing the angle degree (either positively or negatively) of up to about 25 degrees, with changes of ±1°, 2.5°, 5°, 7.5°, and 10° being particularly preferred. b can be changed up to ±1 Å. For β sandwich structures (FIG. 6C and 6D), Ω can be altered up to ±45°, with changes of ±1°, 2.5°, 5°, 7.5°, and 10° being particularly preferred. Similarly, h can be altered as outlined above for α/β proteins, and θ and Φ can be altered up to +30°.

Once the desired value changes are selected, the coordinate positions for the positions chosen are altered to reflect the change, to form a “new” or “altered” backbone protein structure, i.e. one that has all or part of the backbone atoms in a different position relative to the starting structure. It should be noted that this process can be repeated, i.e. additional backbone changes can be made, on the same or different residues. In addition, after optimization, the backbone of one or more optimal sequences can altered and an optimization can be run.

Alternatively, movement of the backbone can be done manually, i.e. sections of the protein backbone can be randomly or arbitrarily moved. In this embodiment, the backbone atoms of one or more amino acids can be moved some distance, generally an angstrom or more, in any direction. This can be done using standard modeling programs; for example, Molecular Dynamics minimization, Monte Carlo dynamics, or random backbone coordinate/angle motion. It is also possible to move the backbone atoms of single residues, that are either components of secondary structural elements or not.

Once a protein structure backbone is generated (with alterations, as outlined above) and input into the computer, explicit hydrogens are added if not included within the structure (for example, if the structure was generated by X-ray crystallography, hydrogens must be added). After hydrogen addition, energy minimization of the structure is run, to relax the hydrogens as well as the other atoms, bond angles and bond lengths. In a preferred embodiment, this is done by doing a number of steps of conjugate gradient minimization (Mayo et al., J. Phys. Chem. 94:8897 (1990)) of atomic coordinate positions to minimize the Dreiding force field with no electrostatics. Generally from about 10 to about 250 steps is preferred, with about 50 being most preferred.

The protein backbone structure contains at least one variable residue position. As is known in the art, the residues, or amino acids, of proteins are generally sequentially numbered starting with the N-terminus of the protein. Thus a protein having a methionine at it's N-terminus is said to have a methionine at residue or amino acid position 1, with the next residues as 2, 3, 4, etc. At each position, the wild type (i.e. naturally occurring) protein may have one of at least 20 amino acids, in any number of rotamers. By “variable residue position” herein is meant an amino acid position of the protein to be designed that is not fixed in the design method as a specific residue or rotamer, generally the wild-type residue or rotamer.

In a preferred embodiment, all of the residue positions of the protein are variable. That is, every amino acid side chain may be altered in the methods of the present invention. This is particularly desirable for smaller proteins, although the present methods allow the design of larger proteins as well. While there is no theoretical limit to the length of the protein which may be designed this way, there is a practical computational limit.

In an alternate preferred embodiment, only some of the residue positions of the protein are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable (for example, to eliminate a proteolytic site or alter the substrate specificity of an enzyme), the residue may be fixed as a particular amino acid. Alternatively, the methods of the present invention may be used to evaluate mutations de novo, as is discussed below. In an alternate preferred embodiment, a fixed position may be “floated”; the amino acid at that position is fixed, but different rotamers of that amino acid are tested. In this embodiment, the variable residues may be at least one, or anywhere from 0.1% to 99.9% of the total number of residues. Thus, for example, it may be possible to change only a few (or one) residues, or most of the residues, with all possibilities in between.

In a preferred embodiment, residues which can be fixed include, but are not limited to, structurally or biologically functional residues. For example, residues which are known to be important for biological activity, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, metal binding sites, critical hydrogen bonding residues, residues critical for backbone conformation such as proline or glycine, residues critical for packing interactions, etc. may all be fixed in a conformation or as a single rotamer, or “floated”.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

As will be appreciated by those in the art, the methods of the present invention allow computational testing of “site-directed mutagenesis” targets without actually making the mutants, or prior to making the mutants. That is, quick analysis of sequences in which a small number of residues are changed can be done to evaluate whether a proposed change is desirable. In addition, this may be done on a known protein, or on an protein optimized as described herein.

As will be appreciated by those in the art, a domain of a larger protein may essentially be treated as a small independent protein; that is, a structural or functional domain of a large protein may have minimal interactions with the remainder of the protein and may essentially be treated as if it were autonomous. In this embodiment, all or part of the residues of the domain may be variable.

It should be noted that even if a position is chosen as a variable position, it is possible that the methods of the invention will optimize the sequence in such a way as to select the wild type residue at the variable position. This generally occurs more frequently for core residues, and less regularly for surface residues. In addition, it is possible to fix residues as non-wild type amino acids as well.

Once the protein backbone structure has been selected and input, and the variable residue positions chosen, a group of potential rotamers for each of the variable residue positions is established. This operation is shown as step 52 in FIG. 2. This step may be implemented using the side chain module 32. In one embodiment of the invention, the side chain module 32 includes at least one rotamer library, as described below, and program code that correlates the selected protein backbone structure with corresponding information in the rotamer library. Alternatively, the side chain module 32 may be omitted and the potential rotamers 42 for the selected protein backbone structure may be downloaded through the input/output devices 26.

As is known in the art, each amino acid side chain has a set of possible conformers, called rotamers. See Ponder, et al., Acad. Press Inc. (London) Ltd. pp. 775-791 (1987); Dunbrack, et al., Struc. Biol. 1(5):334-340 (1994); Desmet, et al., Nature 356:539-542 (1992), all of which are hereby expressly incorporated by reference in their entirety. Thus, a set of discrete rotamers for every amino acid side chain is used. There are two general types of rotamer libraries: backbone dependent and backbone independent. A backbone dependent rotamer library allows different rotamers depending on the position of the residue in the backbone; thus for example, certain leucine rotamers are allowed if the position is within an a helix, and different leucine rotamers are allowed if the position is not in a α-helix. A backbone independent rotamer library utilizes all rotamers of an amino acid at every position. In general, a backbone independent library is preferred in the consideration of core residues, since flexibility in the core is important. However, backbone independent libraries are computationally more expensive, and thus for surface and boundary positions, a backbone dependent library is preferred. However, either type of library can be used at any position.

In addition, a preferred embodiment does a type of “fine tuning” of the rotamer library by expanding the possible χ (chi) angle values of the rotamers by plus and minus one standard deviation (or more) about the mean value, in order to minimize possible errors that might arise from the discreteness of the library. This is particularly important for aromatic residues, and fairly important for hydrophobic residues, due to the increased requirements for flexibility in the core and the rigidity of aromatic rings; it is not as important for the other residues. Thus a preferred embodiment expands the χ₁ and χ₂ angles for all amino acids except Met, Arg and Lys.

To roughly illustrate the numbers of rotamers, in one version of the Dunbrack & Karplus backbone-dependent rotamer library, alanine has 1 rotamer, glycine has 1 rotamer, arginine has 55 rotamers, threonine has 9 rotamers, lysine has 57 rotamers, glutamic acid has 69 rotamers, asparagine has 54 rotamers, aspartic acid has 27 rotamers, tryptophan has 54 rotamers, tyrosine has 36 rotamers, cysteine has 9 rotamers, glutamine has 69 rotamers, histidine has 54 rotamers, valine has 9 rotamers, isoleucine has 45 rotamers, leucine has 36 rotamers, methionine has 21 rotamers, serine has 9 rotamers, and phenylalanine has 36 rotamers.

In general, proline is not generally used, since it will rarely be chosen for any position, although it can be included if desired. Similarly, a preferred embodiment omits cysteine as a consideration, only to avoid potential disulfide problems, although it can be included if desired.

As will be appreciated by those in the art, other rotamer libraries with all dihedral angles staggered can be used or generated.

In a preferred embodiment, at a minimum, at least one variable position has rotamers from at least two different amino acid side chains; that is, a sequence is being optimized, rather than a structure.

In a preferred embodiment, rotamers from all of the amino acids (or all of them except cysteine, glycine and proline) are used for each variable residue position; that is, the group or set of potential rotamers at each variable position is every possible rotamer of each amino acid. This is especially preferred when the number of variable positions is not high as this type of analysis can be computationally expensive.

In a preferred embodiment, each variable position is classified as either a core, surface or boundary residue position, although in some cases, as explained below, the variable position may be set to glycine to minimize backbone strain.

It should be understood that quantitative protein design or optimization studies prior to the present invention focused almost exclusively on core residues. The present invention, however, provides methods for designing proteins containing core, surface and boundary positions. Alternate embodiments utilize methods for designing proteins containing core and surface residues, core and boundary residues, and surface and boundary residues, as well as core residues alone (using the scoring functions of the present invention), surface residues alone, or boundary residues alone.

The classification of residue positions as core, surface or boundary may be done in several ways, as will be appreciated by those in the art. In a preferred embodiment, the classification is done via a visual scan of the original protein backbone structure, including the side chains, and assigning a classification based on a subjective evaluation of one skilled in the art of protein modelling. Alternatively, a preferred embodiment utilizes an assessment of the orientation of the Cα-Cβ vectors relative to a solvent accessible surface computed using only the template Cα atoms. In a preferred embodiment, the solvent accessible surface for only the Cα atoms of the target fold is generated using the Connolly algorithm with a add-on radius ranging from about 4 to about 12 Å, with from about 6 to about 10 Å being preferred, and 8 Å being particularly preferred. The Cα radius used ranges from about 1.6 Å to about 2.3 Å, with from about 1.8 to about 2.1 Å being preferred, and 1.95 Å being especially preferred. A residue is classified as a core position if a) the distance for its Cα, along its Cα-Cβ vector, to the solvent accessible surface is greater than about 4-6 Å, with greater than about 5.0 Å being especially preferred, and b) the distance for its Cβ to the nearest surface point is greater than about 1.5-3 Å, with greater than about 2.0 Å being especially preferred. The remaining residues are classified as surface positions if the sum of the distances from their Cα, along their Cα-Cβ vector, to the solvent accessible surface, plus the distance from their Cβ to the closest surface point was less than about 2.5-4 Å, with less than about 2.7 Å being especially preferred. All remaining residues are classified as boundary positions.

Once each variable position is classified as either core, surface or boundary, a set of amino acid side chains, and thus a set of rotamers, is assigned to each position. That is, the set of possible amino acid side chains that the program will allow to be considered at any particular position is chosen. Subsequently, once the possible amino acid side chains are chosen, the set of rotamers that will be evaluated at a particular position can be determined. Thus, a core residue will generally be selected from the group of hydrophobic residues consisting of alanine, valine, isoleucine, leucine, phenylalanine, tyrosine, tryptophan, and methionine (in some embodiments, when the a scaling factor of the van der Waals scoring function, described below, is low, methionine is removed from the set), and the rotamer set for each core position potentially includes rotamers for these eight amino acid side chains (all the rotamers if a backbone independent library is used, and subsets if a rotamer dependent backbone is used). Similarly, surface positions are generally selected from the group of hydrophilic residues consisting of alanine, serine, threonine, aspartic acid, asparagine, glutamine, glutamic acid, arginine, lysine and histidine. The rotamer set for each surface position thus includes rotamers for these ten residues. Finally, boundary positions are generally chosen from alanine, serine, threonine, aspartic acid, asparagine, glutamine, glutamic acid, arginine, lysine histidine, valine, isoleucine, leucine, phenylalanine, tyrosine, tryptophan, and methionine. The rotamer set for each boundary position thus potentially includes every rotamer for these seventeen residues (assuming cysteine, glycine and proline are not used, although they can be).

Thus, as will be appreciated by those in the art, there is a computational benefit to classifying the residue positions, as it decreases the number of calculations. It should also be noted that there may be situations where the sets of core, boundary and surface residues are altered from those described above; for example, under some circumstances, one or more amino acids is either added or subtracted from the set of allowed amino acids. For example, some proteins which dimerize or multimerize, or have ligand binding sites, may contain hydrophobic surface residues, etc. In addition, residues that do not allow helix “capping” or the favorable interaction with an α-helix dipole may be subtracted from a set of allowed residues. This modification of amino acid groups is done on a residue by residue basis.

In a preferred embodiment, proline, cysteine and glycine are not included in the list of possible amino acid side chains, and thus the rotamers for these side chains are not used. However, in a preferred embodiment, when the variable residue position has a φ angle (that is, the dihedral angle defined by 1) the carbonyl carbon of the preceding amino acid; 2) the nitrogen atom of the current residue; 3) the α-carbon of the current residue; and 4) the carbonyl carbon of the current residue) greater than 0°, the position is set to glycine to minimize backbone strain.

Once the group of potential rotamers is assigned for each variable residue position, processing proceeds to step 54 of FIG. 2. This processing step entails analyzing interactions of the rotamers with each other and with the protein backbone to generate optimized protein sequences. The ranking module 34 may be used to perform these operations. That is, computer code is written to implement the following functions. Simplistically, as is generally outlined above, the processing initially comprises the use of a number of scoring functions, described below, to calculate energies of interactions of the rotamers, either to the backbone itself or other rotamers.

The scoring functions include a Van der Waals potential scoring function, a hydrogen bond potential scoring function, an atomic salvation scoring function, a secondary structure propensity scoring function and an electrostatic scoring function. As is further described below, at least one scoring function is used to score each position, although the scoring functions may differ depending on the position classification or other considerations, like favorable interaction with an α-helix dipole. As outlined below, the total energy which is used in the calculations is the sum of the energy of each scoring function used at a particular position, as is generally shown in Equation 1:

Eₜₒₜₐₗ=nE_{vdw}+nEₐₛ+nE_{h-bonding}+nEₛₛ+nE_{elec} Equation 1

In Equation 1, the total energy is the sum of the energy of the van der Waals potential (E_{vdw}), the energy of atomic solvation (Eₐₛ), the energy of hydrogen bonding (E_{h-bonding}), the energy of secondary structure (Eₛₛ) and the energy of electrostatic interaction (E_{elec}). The term n is either 0 or 1, depending on whether the term is to be considered for the particular residue position, as is more fully outlined below.

In a preferred embodiment, a van der Waals' scoring function is used. As is known in the art, van der Waals' forces are the weak, non-covalent and non-ionic forces between atoms and molecules, that is, the induced dipole and electron repulsion (Pauli principle) forces.

The van der Waals scoring function is based on a van der Waals potential energy. There are a number of van der Waals potential energy calculations, including a Lennard-Jones 12/6 potential with radii and well depth parameters from the Dreiding force field, Mayo et al., J. Prot. Chem., 1990, expressly incorporated herein by reference, or the exponential 6 potential. Equation 2, shown below, is the preferred Lennard-Jones potential:

$${E}_{vdw} = {D}_{0} \{ {( \frac{{R}_{0}}{R} )}^{12} - 2 {( \frac{{R}_{0}}{R} )}^{6} \} \qquad \text{Equation 2}$$

R₀ is the geometric mean of the van der Waals radii of the two atoms under consideration, and D₀ is the geometric mean of the well depth of the two atoms under consideration. E_{vdw} and R are the energy and interatomic distance between the two atoms under consideration, as is more fully described below.

In a preferred embodiment, the van der Waals forces are scaled using a scaling factor, α. Equation 3 shows the use of a in the van der Waals Lennard-Jones potential equation:

$${E}_{vdw} = {D}_{0} \{ {( \frac{α {R}_{0}}{R} )}^{12} - 2 {( \frac{α {R}_{0}}{R} )}^{6} \} \qquad Equation 3$$

The role of the α scaling factor is to change the importance of packing effects in the optimization and design of any particular protein. Specifically, a reduced van der Waals steric constraint can compensate for the restrictive effect of a fixed backbone and discrete side-chain rotamers in the simulation and can allow a broader sampling of sequences compatible with a desired fold. In a preferred embodiment, a values ranging from about 0.70 to about 1.10 can be used, with α values from about 0.8 to about 1.05 being preferred, and from about 0.85 to about 1.0 being especially preferred. Specific a values which are preferred are 0.80, 0.85, 0.90, 0.95, 1.00, and 1.05.

Generally speaking, variation of the van der Waals scale factor a results in four regimes of packing specificity: regime 1 where 0.9≦α≦1.05 and packing constraints dominate the sequence selection; regime 2 where 0.8≦α≦0.9 and the hydrophobic solvation potential begins to compete with packing forces; regime 3 where α<0.8 and hydrophobic solvation dominates the design; and, regime 4 where α>1.05 and van der Waals repulsions appear to be too severe to allow meaningful sequence selection. In particular, different a values may be used for core, surface and boundary positions, with regimes 1 and 2 being preferred for core residues, regime 1 being preferred for surface residues, and regime 1 and 2 being preferred for boundary residues.

In a preferred embodiment, the van der Waals scaling factor is used in the total energy calculations for each variable residue position, including core, surface and boundary positions.

In a preferred embodiment, an atomic salvation potential scoring function is used. As is appreciated by those in the art, solvent interactions of a protein are a significant factor in protein stability, and residue/protein hydrophobicity has been shown to be the major driving force in protein folding. Thus, there is an entropic cost to solvating hydrophobic surfaces, in addition to the potential for misfolding or aggregation. Accordingly, the burial of hydrophobic surfaces within a protein structure is beneficial to both folding and stability. Similarly, there can be a disadvantage for burying hydrophilic residues. The accessible surface area of a protein atom is generally defined as the area of the surface over which a water molecule can be placed while making van der Waals contact with this atom and not penetrating any other protein atom. Thus, in a preferred embodiment, the solvation potential is generally scored by taking the total possible exposed surface area of the moiety or two independent moieties (either a rotamer or the first rotamer and the second rotamer), which is the reference, and subtracting out the “buried” area, i.e. the area which is not solvent exposed due to interactions either with the backbone or with other rotamers. This thus gives the exposed surface area.

Alternatively, a preferred embodiment calculates the scoring function on the basis of the “buried” portion; i.e. the total possible exposed surface area is calculated, and then the calculated surface area after the interaction of the moieties is subtracted, leaving the buried surface area. A particularly preferred method does both of these calculations.

As is more fully described below, both of these methods can be done in a variety of ways. See Eisenberg et al., Nature 319:199-203 (1986); Connolly, Science 221:709-713 (1983); and Wodak, et al., Proc. Natl. Acad. Sci. USA 77(4):1736-1740 (1980), all of which are expressly incorporated herein by reference. As will be appreciated by those in the art, this salvation potential scoring function is conformation dependent, rather than conformation independent.

In a preferred embodiment, the pairwise salvation potential is implemented in two components, “singles” (rotamer/template) and “doubles” (rotamer/rotamer), as is more fully described below. For the rotamer/template buried area, the reference state is defined as the rotamer in question at residue position i with the backbone atoms only of residues i-1, i and i+1, although in some instances just i may be used. Thus, in a preferred embodiment, the salvation potential is not calculated for the interaction of each backbone atom with a particular rotamer, although more may be done as required. The area of the side chain is calculated with the backbone atoms excluding solvent but not counted in the area. The folded state is defined as the area of the rotamer in question at residue i, but now in the context of the entire template structure including non-optimized side chains, i.e. every other fixed position residue. The rotamer/template buried area is the difference between the reference and the folded states. The rotamer/rotamer reference area can be done in two ways; one by using simply the sum of the areas of the isolated rotamers; the second includes the full backbone. The folded state is the area of the two rotamers placed in their relative positions on the protein scaffold but with no template atoms present. In a preferred embodiment, the Richards definition of solvent accessible surface area (Lee and Richards, J. Mol. Biol. 55:379-400, 1971, hereby incorporated by reference) is used, with a probe radius ranging from 0.8 to 1.6 Å, with 1.4 Å being preferred, and Drieding van der Waals radii, scaled from 0.8 to 1.0. Carbon and sulfur, and all attached hydrogens, are considered nonpolar. Nitrogen and oxygen, and all attached hydrogens, are considered polar. Surface areas are calculated with the Connolly algorithm using a dot density of 10 Å-2 (Connolly, (1983) (supra), hereby incorporated by reference).

In a preferred embodiment, there is a correction for a possible overestimation of buried surface area which may exist in the calculation of the energy of interaction between two rotamers (but not the interaction of a rotamer with the backbone). Since, as is generally outlined below, rotamers are only considered in pairs, that is, a first rotamer is only compared to a second rotamer during the “doubles” calculations, this may overestimate the amount of buried surface area in locations where more than two rotamers interact, that is, where rotamers from three or more residue positions come together. Thus, a correction or scaling factor is used as outlined below.

The general energy of salvation is shown in Equation 4:

Eₛₐ=f(SA) Equation 4

where Eₛₐ is the energy of salvation, f is a constant used to correlate surface area and energy, and SA is the surface area. This equation can be broken down, depending on which parameter is being evaluated. Thus, when the hydrophobic buried surface area is used, Equation 5 is appropriate:

Eₛₐ=f₁(SA_{buried hydrophobic}) Equation 5

where f₁ is a constant which ranges from about 10 to about 50 cal/mol/Å², with 23 or 26 cal/mol/Å² being preferred. When a penalty for hydrophilic burial is being considered, the equation is shown in Equation 6:

Eₛₐ=f₁(SA_{buried hydrophobic})+f₂(SA_{buried hydrophilic}) Equation 6

where f₂ is a constant which ranges from −50 to −250 cal/mol/Å², with −86 or −100 cal/mol/Å² being preferred. Similarly, if a penalty for hydrophobic exposure is used, equation 7 or 8 may be used:

Eₛₐ=f₁(SA_{buried hydrophobic})+f₃(SA_{exposed hydrophobic}) Equation 7

Eₛₐ=f₁(SA_{buried hydrophobic})+f₂(SA_{buried hydrophilic})+f₃(SA_{exposed hydrophobic})+f₄(SA_{exposed hydrophobic}) Equation 8

In a preferred embodiment, f₃=−f₁.

In one embodiment, backbone atoms are not included in the calculation of surface areas, and values of 23 cal/mol/Å² (f₁) and −86 cal/mol/Å² (f₂) are determined.

In a preferred embodiment, this overcounting problem is addressed using a scaling factor that compensates for only the portion of the expression for pairwise area that is subject to over-counting. In this embodiment, values of −26 cal/mol/Å² (f₁) and 100 cal/mol/Å² (f₂) are determined.

Atomic solvation energy is expensive, in terms of computational time and resources. Accordingly, in a preferred embodiment, the solvation energy is calculated for core and/or boundary residues, but not surface residues, with both a calculation for core and boundary residues being preferred, although any combination of the three is possible.

In a preferred embodiment, an atomic solvation potential function modified to generate only singles energies is used. This one-body approximation to the atomic solvation potential can be computed in several ways including, but not limited to, using Monte Carlo (MC), mean-field, and approximate dead-end elimination methods. In a preferred embodiment, each rotamer allowed in the design calculation is subjected to a MC simulation such that the rotamer of interest is forced to be in the final solution. This solution is then used to determine the buried and exposed surface areas for the rotamer in question. The one-body atomic salvation potential is computed using these surface areas and is added to the other singles energies for the rotamer in question. This method completely replaces the need to compute rotamer-backbone and rotamer-rotamer solvation energies. Computing rotamer salvation energies in this way allows all of a rotamer's solvation energy to be included in its singles energy at the cost of using a potentially inaccurate representation of the protein surface in the region of the rotamer (that is, a surface that is generated by a MC simulation).

In a preferred embodiment, a hydrogen bond potential scoring function is used. A hydrogen bond potential is used as predicted hydrogen bonds do contribute to designed protein stability (see Stickle et al., J. Mol. Biol. 226:1143 (1992); Huyghues-Despointes et al., Biochem. 34:13267 (1995), both of which are expressly incorporated herein by reference). As outlined previously, explicit hydrogens are generated on the protein backbone structure.

In a preferred embodiment, the hydrogen bond potential consists of a distance-dependent term and an angle-dependent term, as shown in Equation 9:

$${E}_{H \text{-} Bonding} = {D}_{0} \{ 5 {( \frac{{R}_{0}}{R} )}^{12} - 6 {( \frac{{R}_{0}}{R} )}^{10} \} F ( θ , φ , ϕ ) \qquad \text{Equation 9}$$

where R₀ (2.8 Å) and D₀ (8 kcal/mol) are the hydrogen-bond equilibrium distance and well-depth, respectively, and R is the donor to acceptor distance. This hydrogen bond potential is based on the potential used in DREIDING with more restrictive angle-dependent terms to limit the occurrence of unfavorable hydrogen bond geometries. The angle term varies depending on the hybridization state of the donor and acceptor, as shown in Equations 10, 11, 12 and 13. Equation 10 is used for sp³ donor to sp³ acceptor; Equation 11 is used for sp³ donor to sp² acceptor, Equation 12 is used for sp² donor to sp³ acceptor, and Equation 13 is used for sp² donor to sp² acceptor:

F=cos² θ cos²(Φ−109.5) Equation 10

F=cos² θ cos²(Φ) Equation 11

F=cos⁴ θ Equation 12

F=cos² θ cos²(max [Φ,φ]) Equation 13

In Equations 10-13, θ is the donor-hydrogen-acceptor angle, Φ is the hydrogen-acceptor-base angle (the base is the atom attached to the acceptor, for example the carbonyl carbon is the base for a carbonyl oxygen acceptor), and φ is the angle between the normals of the planes defined by the six atoms attached to the sp² centers (the supplement of φ is used when φ is less than 90°). The hydrogen-bond function is only evaluated when 2.6 Å≦R≦3.2 Å, θ>90°, Φ−109.5°<90 ° for the sp³ donor−sp³ acceptor case, and, Φ>90° for the sp³ donor−sp² acceptor case; preferably, no switching functions are used. Template donors and acceptors that are involved in template-template hydrogen bonds are preferably not included in the donor and acceptor lists. For the purpose of exclusion, a template-template hydrogen bond is considered to exist when 2.5 Å≦R<3.3 Å and θ≧135°.

The hydrogen-bond potential may also be combined or used with a weak coulombic term that includes a distance-dependent dielectric constant of 40R, where R is the interatomic distance. Partial atomic charges are preferably only applied to polar functional groups. A net formal charge of +1 is used for Arg and Lys and a net formal charge of −1 is used for Asp and Glu; see Gasteiger, et al., Tetrahedron 36:3219-3288 (1980); Rappe, et al., J. Phys. Chem. 95:3358-3363 (1991).

In a preferred embodiment, an explicit penalty is given for buried polar hydrogen atoms which are not hydrogen bonded to another atom. See Eisenberg, et al., (1986) (supra), hereby expressly incorporated by reference. In a preferred embodiment, this penalty for polar hydrogen burial, is from about 0 to about 3 kcal/mol, with from about 1 to about 3 being preferred and 2 kcal/mol being particularly preferred. This penalty is only applied to buried polar hydrogens not involved in hydrogen bonds. A hydrogen bond is considered to exist when E_{HB} ranges from about 1 to about 4 kcal/mol, with E_{HB} of less than −2 kcal/mol being preferred. In addition, in a preferred embodiment, the penalty is not applied to template hydrogens, i.e. unpaired buried hydrogens of the backbone.

In a preferred embodiment, only hydrogen bonds between a first rotamer and the backbone are scored, and rotamer-rotamer hydrogen bonds are not scored. In an alternative embodiment, hydrogen bonds between a first rotamer and the backbone are scored, and rotamer-rotamer hydrogen bonds are scaled by 0.5.

In a preferred embodiment, the hydrogen bonding scoring function is used for all positions, including core, surface and boundary positions. In alternate embodiments, the hydrogen bonding scoring function may be used on only one or two of these.

In a preferred embodiment, a secondary structure propensity scoring function is used. This is based on the specific amino acid side chain, and is conformation independent. That is, each amino acid has a certain propensity to take on a secondary structure, either α-helix or β-sheet, based on its Φ and ψ angles. See Muñoz et al., Current Op. in Biotech. 6:382 (1995); Minor, et al., Nature 367:660-663 (1994); Padmanabhan, et al., Nature 344:268-270 (1990); Muñoz, et al., Folding & Design 1(3):167-178 (1996); and Chakrabartty, et al., Protein Sci. 3:843 (1994), all of which are expressly incorporated herein by reference. Thus, for variable residue positions that are in recognizable secondary structure in the backbone, a secondary structure propensity scoring function is preferably used. That is, when a variable residue position is in an α-helical area of the backbone, the α-helical propensity scoring function described below is calculated. Whether or not a position is in a α-helical area of the backbone is determined as will be appreciated by those in the art, generally on the basis of Φ and ψ angles; for α-helix, Φ angles from −2 to −70 and ψ angles from −30 to −100 generally describe an α-helical area of the backbone.

Similarly, when a variable residue position is in a β-sheet backbone conformation, the β-sheet propensity scoring function is used. β-sheet backbone conformation is generally described by Φ angles from −30 to −100 and χ angles from +40 to +180. In alternate preferred embodiments, variable residue positions which are within areas of the backbone which are not assignable to either β-sheet or α-helix structure may also be subjected to secondary structure propensity calculations.

In a preferred embodiment, energies associated with secondary propensities are calculated using Equation 14:

E_{∝}=10^{N}^{ₛₛ}^{(ΔG°}^{ₐₐ}^{-ΔG°}^{ₐₗₐ}⁾−1) Equation 14

In Equation 14, E_{α}(or E_{β}) is the energy of α-helical propensity, ΔG°ₐₐ is the standard free energy of helix propagation of the amino acid, and ΔG°ₐₗₐ, is the standard free energy of helix propagation of alanine used as a standard, or standard free energy of β-sheet formation of the amino acid, both of which are available in the literature (see Chakrabartty, et al., (1994) (supra), and Munoz, et al., (1996) (supra)), both of which are expressly incorporated herein by reference), and Nₛₛ is the propensity scale factor which is set to range from 1 to 4, with 2.0 being preferred. This potential is preferably selected in order to scale the propensity energies to a similar range as the other terms in the scoring function.

In a preferred embodiment, β-sheet propensities are preferably calculated only where the i−1 and i+1 residues are also in β-sheet conformation.

In a preferred embodiment, the secondary structure propensity scoring function is used only in the energy calculations for surface variable residue positions. In alternate embodiments, the secondary structure propensity scoring function is used in the calculations for core and boundary regions as well.

In a preferred embodiment, an electrostatic scoring function is used, as shown below in Equation 15:

$${E}_{elec} = \frac{{qq}^{′}}{ϵ {r}^{2}} \qquad \text{Equation 15}$$

In this Equation, q is the charge on atom 1, q′ is charge on atom 2, and r is the interaction distance.

In a preferred embodiment, a rotamer internal strain energy potential scoring function is used. As appreciated by those of skill in the art, for a given rotamer, a number of different internal interactions may occur between different atoms that contribute to designed protein stability. The contribution of these internal interactions may be determined by using one or more of the following scoring functions: 1) van der Waals scoring function; 2) Coulumbic potential; 3) hydrogen bond potential; and d) torsional potential.

In a preferred embodiment, at least one scoring function is used for each variable residue position; in preferred embodiments, two, three or four scoring functions are used for each variable residue position.

Once the scoring functions to be used are identified for each variable position, the preferred first step in the computational analysis comprises the determination of the interaction of each possible rotamer with all or part of the remainder of the protein. That is, the energy of interaction, as measured by one or more of the scoring functions, of each possible rotamer at each variable residue position with either the backbone or other rotamers, is calculated. In a preferred embodiment, the interaction of each rotamer with the entire remainder of the protein, i.e. both the entire template and all other rotamers, is done. However, as outlined above, it is possible to only model a portion of a protein, for example a domain of a larger protein, and thus in some cases, not all of the protein need be considered.

In a preferred embodiment, the first step of the computational processing is done by calculating two sets of interactions for each rotamer at every position (FIG. 3A): the interaction of the rotamer side chain with the template or backbone (the “singles” energy), and the interaction of the rotamer side chain with all other possible rotamers at every other position (the “doubles” energy), whether that position is varied or floated. It should be understood that the backbone in this case includes both the atoms of the protein structure backbone, as well as the atoms of any fixed residues, wherein the fixed residues are defined as a particular conformation of an amino acid.

Thus, “singles” (rotamer/template) energies are calculated for the interaction of every possible rotamer at every variable residue position with the backbone, using some or all of the scoring functions. Thus, for the hydrogen bonding scoring function, every hydrogen bonding atom of the rotamer and every hydrogen bonding atom of the backbone is evaluated, and the E_{HB} is calculated for each possible rotamer at every variable position. Similarly, for the van der Waals scoring function, every atom of the rotamer is compared to every atom of the template (generally excluding the backbone atoms of its own residue), and the E_{vdW} is calculated for each possible rotamer at every variable residue position. In addition, generally no van der Waals energy is calculated if the atoms are connected by three bonds or less. For the atomic solvation scoring function, the surface of the rotamer is measured against the surface of the template, and the Eₐₛ for each possible rotamer at every variable residue position is calculated. The secondary structure propensity scoring function is also considered as a singles energy, and thus the total singles energy may contain an Eₛₛ term. As will be appreciated by those in the art, many of these energy terms will be close to zero, depending on the physical distance between the rotamer and the template position; that is, the farther apart the two moieties, the closer to zero.

Accordingly, as outlined above, the total singles energy is the sum of the energy of each scoring function used at a particular position, as shown in Equation 1, wherein n is either 1 or zero, depending on whether that particular scoring function was used at the rotamer position:

Eₜₒₜₐₗ=nE_{vdw}+nEₐₛ+nE_{h-bonding}+nEₛₛ+nE_{elec} Equation 1

Once calculated, each singles Eₜₒₜₐₗ for each possible rotamer is stored in the memory 24 within the computer, such that it may be used in subsequent calculations, as outlined below.

For the calculation of “doubles” energy (rotamerirotamer), the interaction energy of each possible rotamer is compared with every possible rotamer at all other variable residue positions. Thus, “doubles” energies are calculated for the interaction of every possible rotamer at every variable residue position with every possible rotamer at every other variable residue position, using some or all of the scoring functions. Thus, for the hydrogen bonding scoring function, every hydrogen bonding atom of the first rotamer and every hydrogen bonding atom of every possible second rotamer is evaluated, and the E_{HB} is calculated for each possible rotamer pair for any two variable positions. Similarly, for the van der Waals scoring function, every atom of the first rotamer is compared to every atom of every possible second rotamer, and the E_{vdW} is calculated for each possible rotamer pair at every two variable residue positions. For the atomic salvation scoring function, the surface of the first rotamer is measured against the surface of every possible second rotamer, and the Eₛₛ for each possible rotamer pair at every two variable residue positions is calculated. The secondary structure propensity scoring function need not be run as a “doubles” energy, as it is considered as a component of the “singles” energy. As will be appreciated by those in the art, many of these double energy terms will be close to zero, depending on the physical distance between the first rotamer and the second rotamer; that is, the farther apart the two moieties, the closer to zero.

Accordingly, as outlined above, the total doubles energy is the sum of the energy of each scoring function used to evaluate every possible pair of rotamers, as shown in Equation 16, wherein n is either 1 or zero, depending on whether that particular scoring function was used at the rotamer position:

Eₜₒₜₐₗ=nE_{vdw}+nEₐₛ+nE_{h-bonding}+E_{elec} Equation 16

An example is illuminating. A first variable position, i, has three (an unrealistically low number) possible rotamers (which may be either from a single amino acid or different amino acids) which are labelled iₐ, i_{b}, and i_{c}. A second variable position, j, also has three possible rotamers, labelled j_{d}, jₑ, and j_{f}. Thus, nine doubles energies (Eₜₒₜₐₗ) are calculated in all: Eₜₒₜₐₗ(iₐ, j_{d}), Eₜₒₜₐₗ(iₐ, ⱼₑ), Eₜₒₜₐₗ(iₐ, j_{f}), Eₜₒₜₐₗ(i_{b}, j_{d}), Eₜₒₜₐₗ(i_{b}, jₑ), Eₜₒₜₐₗ(i_{b}, j_{f}), Eₜₒₜₐₗ(i_{c}, j_{d}), Eₜₒₜₐₗ(i_{c}, jₑ), and Eₜₒₜₐₗ(i_{c}, j_{f}).

Once calculated, each doubles Eₜₒₜₐₗ for each possible rotamer pair is stored in memory 24 within the computer, such that it may be used in subsequent calculations, as outlined below.

Once the singles and doubles energies are calculated and stored, the next step of the computational processing may occur. Generally speaking, the goal of the computational processing is to determine a set of optimized protein sequences. By “optimized protein sequence” herein is meant a sequence that best fits the mathematical equations herein. As will be appreciated by those in the art, a global optimized sequence is the one sequence that best fits Equation 1, i.e. the sequence that has the lowest energy of any possible sequence. However, there are any number of sequences that are not the global minimum but that have low energies.

In a preferred embodiment, the set comprises the globally optimal sequence in its optimal conformation, i.e. the optimum rotamer at each variable position. That is, computational processing is run until the simulation program converges on a single sequence which is the global optimum.

In a preferred embodiment, the set comprises at least two optimized protein sequences. Thus for example, the computational processing step may eliminate a number of disfavored combinations but be stopped prior to convergence, providing a set of sequences of which the global optimum is one. In addition, further computational analysis, for example using a different method, may be run on the set, to further eliminate sequences or rank them differently. Alternatively, as is more fully described below, the global optimum may be reached, and then further computational processing may occur, which generates additional optimized sequences in the neighborhood of the global optimum.

If a set comprising more than one optimized protein sequences is generated, they may be rank ordered in terms of theoretical quantitative stability, as is more fully described below.

In a preferred embodiment, the computational processing step first comprises an elimination step, sometimes referred to as “applying a cutoff”, either a singles elimination or a doubles elimination. Singles elimination comprises the elimination of all rotamers with template interaction energies of greater than about 10 kcal/mol prior to any computation, with elimination energies of greater than about 15 kcal/mol being preferred and greater than about 25 kcal/mol being especially preferred. Similarly, doubles elimination is done when a rotamer has interaction energies greater than about 10 kcal/mol with all rotamers at a second residue position, with energies greater than about 15 being preferred and greater than about 25 kcal/mol being especially preferred.

In a preferred embodiment, the computational processing comprises direct determination of total sequence energies, followed by comparison of the total sequence energies to ascertain the global optimum and rank order the other possible sequences, if desired. The energy of a total sequence is shown below in Equation 17:

$${E}_{total protein} = {E}_{( b - b )} + {\sum }_{all i} {E}_{( {i}_{a} )} + {\sum }_{all i} {\sum }_{all j pairs} {E}_{( {i}_{a} , {j}_{a} )} \qquad \text{Equation 17}$$

Thus every possible combination of rotamers may be directly evaluated by adding the backbone-backbone (sometimes referred to herein as template-template) energy (E_{(b-b)} which is constant over all sequences herein since the backbone is kept constant), the singles energy for each rotamer (which has already been calculated and stored), and the doubles energy for each rotamer pair (which has already been calculated and stored). Each total sequence energy of each possible rotamer sequence can then be ranked, either from best to worst or worst to best. This is obviously computationally expensive and becomes unwieldy as the length of the protein increases.

In a preferred embodiment, the computational processing includes one or more Dead-End Elimination (DEE) computational steps. The DEE theorem is the basis for a very fast discrete search program that was designed to pack protein side chains on a fixed backbone with a known sequence. See Desmet, et al., Nature 356:539-542 (1992); Desmet, et al., The Proetin Folding Problem and Tertiary Structure Prediction, Ch. 10:1-49 (1994); Goldstein, Biophys. Jour. 66:1335-1340 (1994), all of which are incorporated herein by reference. DEE is based on the observation that if a rotamer can be eliminated from consideration at a particular position, i.e. make a determination that a particular rotamer is definitely not part of the global optimal conformation, the size of the search is reduced. This is done by comparing the worst interaction (i.e. energy or Eₜₒₜₐₗ) of a first rotamer at a single variable position with the best interaction of a second rotamer at the same variable position. If the worst interaction of the first rotamer is still better than the best interaction of the second rotamer, then the second rotamer cannot possibly be in the optimal conformation of the sequence. The original DEE theorem is shown in Equation 23:

$$E ( {i}_{a} ) + {\sum }_{j} [ min over t \{ E ( {i}_{a} , {j}_{t} ) \} ] > E ( {i}_{b} ) + {\sum }_{j} [ max over t \{ E ( {i}_{b} , {j}_{t} ) \} ] \qquad \text{Equation 23}$$

In Equation 23, rotamer iₐ is being compared to rotamer i_{b} (for notational convenience iₐ, and i_{b} are the equivalents of iᵣ, iₜ, respectively, in Equation 35). The left side of the inequality is the best possible interaction energy (Eₜₒₜₐₗ) of iₐ with the rest of the protein; that is, “min over t” means find the rotamer t on position j that has the best interaction with rotamer iₐ. Similarly, the right side of the inequality is the worst possible (max) interaction energy of rotamer i_{b} with the rest of the protein. If this inequality is true, then rotamer iₐ is Dead-Ending and can be Eliminated. The speed of DEE comes from the fact that the theorem only requires sums over the sequence length to test and eliminate rotamers.

In a preferred embodiment, a variation of DEE is performed. Goldstein DEE, based on Goldstein, (1994) (supra), hereby expressly incorporated by reference, is a variation of the DEE computation, as shown in Equation 24:

E(iₐ)−E(i_{b})+Σ[min over t{E(iₐ, jₜ)−E(i_{b}, jₜ)}]>0 Equation 24

In essence, the Goldstein Equation 24 says that a first rotamer a of a particular position i (rotamer iₐ) will not contribute to a local energy minimum if the energy of conformation with iₐ can always be lowered by just changing the rotamer at that position to i_{b}, keeping the other residues equal (for notational convenience iₐ, and i_{b} are the equivalents of iᵣ, iₜ, respectively, in Equation 36). If this inequality is true, then rotamer iₐ is Dead-Ending and can be Eliminated.

Thus, in a preferred embodiment, a first DEE computation is done where rotamers at a single variable position are compared, (“singles” DEE) to eliminate rotamers at a single position. This analysis is repeated for every variable position, to eliminate as many single rotamers as possible. In addition, every time a rotamer is eliminated from consideration through DEE, the minimum and maximum calculations of Equation 23, depending on which DEE variation is used, thus conceivably allowing the elimination of further rotamers. Accordingly, the singles DEE computation can be repeated until no more rotamers can be eliminated; that is, when the inequality is not longer true such that all of them could conceivably be found on the global optimum.

In a preferred embodiment, “doubles” DEE is additionally done. In doubles DEE, pairs of rotamers are evaluated; that is, a first rotamer at a first position and a second rotamer at a second position are compared to a third rotamer at the first position and a fourth rotamer at the second position, either using original or Goldstein DEE. Pairs are then flagged as nonallowable, although single rotamers cannot be eliminated, only the pair. Again, as for singles DEE, every time a rotamer pair is flagged as nonallowable, the minimum calculations of Equation 24 change (depending on which DEE variation is used) thus conceivably allowing the flagging of further rotamer pairs. Accordingly, the doubles DEE computation can be repeated until no more rotamer pairs can be flagged.

As is apparent from equations (17) and (24), the performance of any DEE algorithm depends heavily on the nature of the physical model used to compute the singles energy (i.e. rotamer-backbone; defined above) and the doubles energy (i.e. rotamer-rotamer; defined above) terms E(iₐ) and E(i_{a,j}ₜ), respectively in equation (17). Potential scoring functions that emphasize energy terms that contribute to E(iₐ) relative to E(i,jₜ) will result in less coupling and easier optimization. In the limit of |E(iₐ)|>>E(iₐ,jₜ)|, the optimization reduces to the selection of the rotamer with the best one-body energy at each residue position.

In a preferred embodiment, optimization with a potential scoring function modified to emphasize singles energy terms is used. Scoring functions that can be modified to favor E(iₐ) include, but are not limited to: (1) atomic solvation potential; (2) Coulombic potential with a non-distance-dependent dielectric constant for rotamer/backbone interactions and a distance-dependent dielectric constant for rotamer/rotamer interactions; (3) rotamer internal strain energy; (4) secondary structure propensities for helical and beta-strand positions; and, (5) van der Waals energies.

In addition, in a preferred embodiment, rotamer pairs are initially prescreened to eliminate rotamer pairs prior to DEE. This is done by doing relatively computationally inexpensive calculations to eliminate certain pairs up front. This may be done in several ways, as is outlined below.

To search exhaustively for all dead-ending rotamers at a residue position i, it is necessary to compare every rotamer to every other rotamer available at i. In a comparison matrix, each column corresponds to a particular rotamer, iᵣ as a candidate r for elimination, and each row corresponds to one of the possible reference rotamers iₜ. If there are n t rotamers at position i, then an exhaustive search of n²−n matrix elements is necessary. Such a matrix is evaluated for each of the positions that may be represented by i.

In a preferred embodiment, the rotamer pair with the lowest interaction energy with the rest of the system is found. Inspection of the energy distributions in sample comparison matrices has revealed that an iᵤjᵥ pair that dead-end eliminates a particular iᵣjₛ pair can also eliminate other iᵣjₛ pairs. In fact, there are often a few iᵤjᵥ pairs, which we call “magic bullets,” that eliminate a significant number of iᵣjₛ pairs. We have found that one of the most potent magic bullets is the pair for which maximum interaction energy, εₘₐₓ([iᵤjᵥ])kₜ, is least (see Equations 29-31). This pair is referred to as (iᵤjᵥ)_{mb}. If this rotamer pair is used in the first round of doubles DEE, it tends to eliminate pairs faster.

Our first speed enhancement is to evaluate the first-order doubles calculation for only the matrix elements in the row corresponding to the (iᵤⱼᵥ)_{mb} pair. The discovery of (iᵤjᵥ)_{mb i}s an n² calculation (n=the number of rotamers per position), and the application of Equation 24 to the single row of the matrix corresponding to this rotamer pair is another n² calculation, so the calculation time is small in comparison to a full Goldstein calculation. In practice, this calculation produces a large number of dead-ending pairs, often enough to proceed to the next iteration of singles elimination without any further searching of the doubles matrix.

The magic bullet Goldstein calculation will also discover all dead-ending pairs that would be discovered by the Equation 23 or 24, thereby making it unnecessary. This stems from the fact that εₘₐₓ(iᵤjᵥ)_{mb} must be less than or equal to any εₘₐₓ(iᵤjᵥ) that would successfully eliminate a pair by Equations 23 or 24.

Since the minima and maxima of any given pair has been precalculated as outlined herein, a second speed-enhancement precalculation may be done. By comparing extrema, pairs that will not dead end can be identified and thus skipped, reducing the time of the DEE calculation. Thus, pairs that satisfy either one of the following criteria are skipped:

εₘᵢₙ([iᵣjₛ])<εₘᵢₙ([iᵤjᵥ]) Equation 25

εₘₐₓ([iᵣjₛ])<εₘₐₓ([iᵤjᵥ]) Equation 26

Because the matrix containing these calculations is symmetrical, half of its elements will satisfy the first inequality Equation 25, and half of those remaining will satisfy the other inequality Equation 26. These three quarters of the matrix need not be subjected to the evaluation of Equation 23 or 24, resulting in a theoretical speed enhancement of a factor of four.

The last DEE speed enhancement refines the search of the remaining quarter of the matrix. This is done by constructing a metric from the precomputed extrema to detect those matrix elements likely to result in a dead-ending pair.

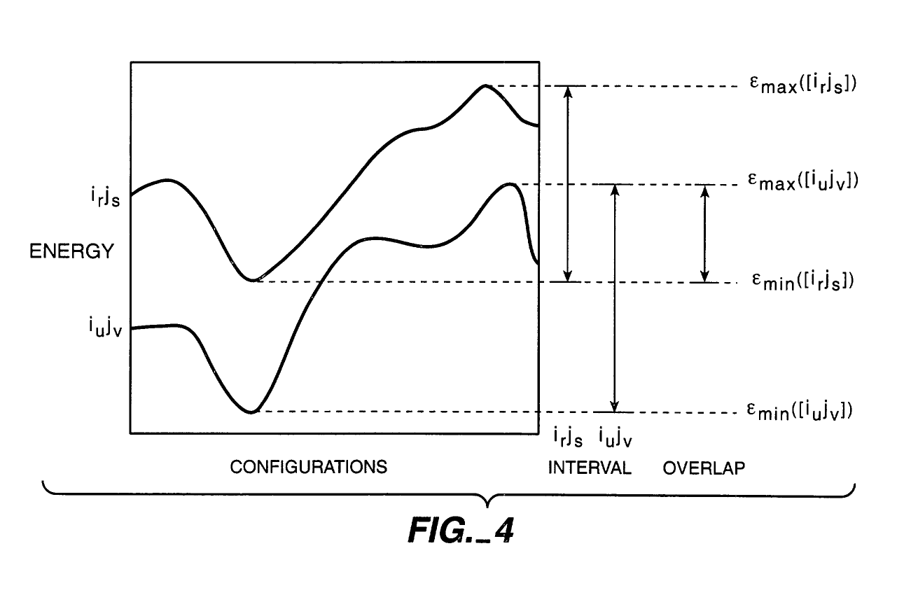

A metric was found through analysis of matrices from different sample optimizations, We searched for combinations of the extrema that predicted the likelihood that a matrix element would produce a dead-ending pair. Interval sizes (see FIG. 4) for each pair were computed from differences of the extrema. The size of the overlap of the iᵣjₛ and iᵤjᵥ intervals were also computed, as well as the difference between the minima and the difference between the maxima. Combinations of these quantities, as well as the lone extrema, were tested for their ability to predict the occurrence of dead-ending pairs. Because some of the maxima were very large, the quantities were also compared logarithmically.

Most of the combinations were able to predict dead-ending matrix elements to varying degrees. The best metrics were the fractional interval overlap with respect to each pair, referred to herein as qᵣₛ and qᵤᵥ.

$$\begin{matrix}{q}_{rs} = \frac{interval overlap}{interval ( [ {i}_{r} {j}_{s} ] )} = \frac{{ϵ}_{max} ( [ {i}_{u} {j}_{v} ] ) - {ϵ}_{min} ( [ {i}_{r} {j}_{s} ] )}{{ϵ}_{max} ( [ {i}_{r} {j}_{s} ] ) - {ϵ}_{min} ( [ {i}_{r} {j}_{s} ] )} & \text{Equation 27} \\ {q}_{uv} = \frac{interval overlap}{interval ( [ {i}_{u} {j}_{v} ] )} = \frac{{ϵ}_{max} ( [ {i}_{u} {j}_{v} ] ) - {ϵ}_{min} ( [ {i}_{r} {j}_{s} ] )}{{ϵ}_{max} ( [ {i}_{u} {j}_{v} ] ) - {ϵ}_{min} ( [ {i}_{u} {j}_{v} ] )} & \text{Equation 28}\end{matrix}$$

These values are calculated using the minima and maxima equations 29, 30, 31 and 32 (see FIG. 5):

$$\begin{matrix}{ϵ}_{max} ( [ {i}_{r} {j}_{s} ] ) = ϵ ( [ {i}_{r} {j}_{s} ] ) + {\sum }_{k \neq i \neq j} {max }_{t} ϵ ( [ {i}_{r} {j}_{s} ] , {k}_{t} & \text{Equation 29} \\ {ϵ}_{min} ( [ {i}_{r} {j}_{s} ] ) = ϵ ( [ {i}_{r} {j}_{s} ] ) + {\sum }_{k \neq i \neq j} {min }_{t} ϵ ( [ {i}_{r} {j}_{s} ] , {k}_{t} ) & \text{Equation 30} \\ {ϵ}_{max} ( [ {i}_{u} {j}_{v} ] ) = ϵ ( [ {i}_{u} {j}_{v} ] ) + {\sum }_{k \neq i \neq j} {max }_{t} ϵ ( [ {i}_{u} {j}_{v} ] , {k}_{t} ) & \text{Equation 31} \\ {ϵ}_{min} ( [ {i}_{u} {j}_{v} ] ) = ϵ ( [ {i}_{u} {j}_{v} ] ) + {\sum }_{k \neq i \neq j} {min }_{t} ϵ ( [ {i}_{u} {j}_{v} ] , {k}_{t} ) & \text{Equation 32}\end{matrix}$$

These metrics were selected because they yield ratios of the occurrence of dead-ending matrix elements to the total occurrence of elements that are higher than any of the other metrics tested. For example, there are very few matrix elements (˜2%) for which qᵣₛ>0.98, yet these elements produce 30-40% of all of the dead-ending pairs.

Accordingly, the first-order doubles criterion is applied only to those doubles for which qᵣₛ>0.98 and qᵤᵥ>0.99. The sample data analyses predict that by using these two metrics, as many as half of the dead-ending elements may be found by evaluating only two to five percent of the reduced matrix.

In a preferred embodiment for practicing automated protein design, simple spit DEE (s=1; Equation 38) is used to find the GMEC.

In a preferred embodiment for practicing automated protein design, split singles DEE (s>1; Equation 39) is used to find the GMEC.

In a preferred embodiment for practicing automated protein design, split singles DEE flags with split flags (s=1) is used to find the GMEC.

In a preferred embodiment, split singles DEE with split flags (s≧2) with or without magic bullet metric (equation 40) is used to find the GMEC.

In a preferred embodiment for practicing automated protein design, magic bullet metric (Equation 40) is used to find the GMEC.

Generally, as is more fully described below, singles and doubles DEE, using either or both of original DEE and Goldstein DEE, simple split DEE (s=1), split singles DEE (s>1), and magic bullet metric, is run iteratively until no further elimination is possible. Usually, convergence is not complete, and further elimination must occur to achieve convergence.

In a preferred embodiment, additional DEE computation is done by the creation of “super residues” or “unification”, as is generally described in Desmet, Nature 356:539-542 (1992); Desmet, et al., The Protein Folding Problem and Tertiary Structure Prediction, Ch. 10:1-49 (1994); Goldstein, et al., supra.

A super residue is a combination of two or more variable residue positions which is then treated as a single residue position. The super residue is then evaluated in singles DEE, and doubles DEE, with either other residue positions or super residues. The disadvantage of super residues is that there are many more rotameric states which must be evaluated; that is, if a first variable residue position has 5 possible rotamers, and a second variable residue position has 4 possible rotamers, there are 20 possible super residue rotamers which must be evaluated. However, these super residues may be eliminated similar to singles, rather than being flagged like pairs.

The selection of which positions to combine into super residues may be done in a variety of ways. In general, random selection of positions for super residues results in inefficient elimination, but it can be done, although this is not preferred. In a preferred embodiment, the first evaluation is the selection of positions for a super residue is the number of rotamers at the position. If the position has too many rotamers, it is never unified into a super residue, as the computation becomes too unwieldy. Thus, only positions with fewer than about 100,000 rotamers are chosen, with less than about 50,000 being preferred and less than about 10,000 being especially preferred.

In a preferred embodiment, the evaluation of whether to form a super residue is done as follows. All possible rotamer pairs are ranked using Equation 33, and the rotamer pair with the highest number is chosen for unification:

$$\frac{( \text{fraction of flagged pairs} )}{log ( \begin{matrix}\text{number of super rotamers resulting} \\ \text{from the potential unification}\end{matrix} )} \qquad \text{Equation 33}$$

Equation 33 is looking for the pair of positions that has the highest fraction or percentage of flagged pairs but the fewest number of super rotamers. That is, the pair that gives the highest value for Equation 33 is preferably chosen. Thus, if the pair of positions that has the highest number of flagged pairs but also a very large number of super rotamers (that is, the number of rotamers at position i times the number of rotamers at position j), this pair may not be chosen (although it could) over a lower percentage of flagged pairs but fewer super rotamers.

In an alternate preferred embodiment, positions are chosen for super residues that have the highest average energy; that is, for positions i and j, the average energy of all rotamers for i and all rotamers for j is calculated, and the pair with the highest average energy is chosen as a super residue.

Super residues are made one at a time, preferably. After a super residue is chosen, the singles and doubles DEE computations are repeated where the super residue is treated as if it were a regular residue. As for singles and doubles DEE, the elimination of rotamers in the super residue DEE will alter the minimum energy calculations of DEE. Thus, repeating singles and/or doubles DEE can result in further elimination of rotamers.

FIG. 3A is a detailed illustration of the processing operations associated with a ranking module 34 of the invention. The calculation and storage of the singles and doubles energies is the first step, although these may be recalculated every time. The optional application of a cutoff, where singles or doubles energies that are too high are eliminated prior to further processing also may be included. Either or both of original singles DEE or Goldstein singles DEE may be done, with the elimination of original singles DEE being generally preferred. Once the singles DEE is run, original doubles and/or Goldstein doubles DEE is run. Super residue DEE is then generally run, either original or Goldstein super residue DEE. This preferably results in convergence at a global optimum sequence. As is depicted in FIG. 3A, after any step any or all of the previous steps can be rerun, in any order.

The addition of super residue DEE to the computational processing, with repetition of the previous DEE steps, generally results in convergence at the global optimum. Convergence to the global optimum is guaranteed if no cutoff applications are made, although generally a global optimum is achieved even with these steps. In a preferred embodiment, DEE is run until the global optimum sequence is found. That is, the set of optimized protein sequences contains a single member, the global optimum.

In a preferred embodiment, the various DEE steps are run until a manageable number of sequences is found, i.e. no further processing is required. These sequences represent a set of optimized protein sequences, and they can be evaluated as is more fully described below. Generally, for computational purposes, a manageable number of sequences depends on the length of the sequence, but generally ranges from about 1 to about 10¹⁵ possible rotamer sequences. This range can be extended to approximately 10³⁰ if B&T is used as the next analyzing step.

Alternatively, DEE is run to a point, resulting in a set of optimized sequences (in this context, a set of remainder sequences) and then further computational processing of a different type may be run. For example, in one embodiment, direct calculation of sequence energy as outlined above is done on the remainder possible sequences. Alternatively, a Monte Carlo search can be run. In another embodiment, B&T can be run.

In a preferred embodiment, the computation processing need not comprise a DEE computational step.

In this embodiment, a Monte Carlo search is undertaken, as is known in the art. See Metropolis et al., J. Chem. Phys. 21:1087 (1953), hereby incorporated by reference. In this embodiment, a random sequence comprising random rotamers is chosen as a start point. In one embodiment, the variable residue positions are classified as core, boundary or surface residues and the set of available residues at each position is thus defined. Then a random sequence is generated, and a random rotamer for each amino acid is chosen. This serves as the starting sequence of the Monte Carlo search. A Monte Carlo search then makes a random jump at one position, either to a different rotamer of the same amino acid or a rotamer of a different amino acid, and then a new sequence energy (E_{total sequence}) is calculated, and if the new sequence energy meets the Boltzmann criteria for acceptance, it is used as the starting point for another jump. If the Boltzmann test fails, another random jump is attempted from the previous sequence. In this way, sequences with lower and lower energies are found, to generate a set of low energy sequences.

If computational processing results in a single global optimum sequence, it is frequently preferred to generate additional sequences in the energy neighborhood of the global solution, which may be ranked. These additional sequences are also optimized protein sequences. The generation of additional optimized sequences is generally preferred so as to evaluate the differences between the theoretical and actual energies of a sequence. Generally, in a preferred embodiment, the set of sequences is at least about 75% homologous to each other, with at least about 80% homologous being preferred, at least about 85% homologous being particularly preferred, and at least about 90% being especially preferred. In some cases, homology as high as 95% to 98% is desirable. Homology in this context means sequence similarity or identity, with identity being preferred. Identical in this context means identical amino acids at corresponding positions in the two sequences which are being compared. Homology in this context includes amino acids which are identical and those which are similar (functionally equivalent). This homology will be determined using standard techniques known in the art, such as the Best Fit sequence program described by Devereux, et al., Nucl. Acid Res., 12:387-395 (1984), or the BLASTX program (Altschul, et al., J. Mol. Biol., 215:403-410 (1990)) preferably using the default settings for either. The alignment may include the introduction of gaps in the sequences to be aligned. In addition, for sequences which contain either more or fewer amino acids than an optimum sequence, it is understood that the percentage of homology will be determined based on the number of homologous amino acids in relation to the total number of amino acids. Thus, for example, homology of sequences shorter than an optimum will be determined using the number of amino acids in the shorter sequence.

Once optimized protein sequences are identified, the processing of FIG. 2 optionally proceeds to step 56 which entails searching the protein sequences. This processing may be implemented with the search module 36. The search module 36 is a set of computer code that executes a search strategy. For example, the search module 36 may be written to execute a Monte Carlo search as described above. Starting with the global solution, random positions are changed to other rotamers allowed at the particular position, both rotamers from the same amino acid and rotamers from different amino acids. A new sequence energy (E_{total sequence}) is calculated, and if the new sequence energy meets the Boltzmann criteria for acceptance, it is used as the starting point for another jump. See Metropolis et a., 1953, supra, hereby incorporated by reference. If the Boltzmann test fails, another random jump is attempted from the previous sequence. A list of the sequences and their energies is maintained during the search. After a predetermined number of jumps, the best scoring sequences may be output as a rank-ordered list. Preferably, at least about 10⁶ jumps are made, with at least about 10⁷ jumps being preferred and at least about 10⁸ jumps being particularly preferred. Preferably, at least about 100 to 1000 sequences are saved, with at least about 10,000 sequences being preferred and at least about 100,000 to 1,000,000 sequences being especially preferred. During the search, the temperature is preferably set to 1000 K.

Once the Monte Carlo search is over, all of the saved sequences are quenched by changing the temperature to 0 K, and fixing the amino acid identity at each position. Preferably, every possible rotamer jump for that particular amino acid at every position is then tried.

The computational processing results in a set of optimized protein sequences. These optimized protein sequences are generally, but not always, significantly different from the wild-type sequence from which the backbone was taken. That is, each optimized protein sequence preferably comprises at least about 5-10% variant amino acids from the starting or wild-type sequence, with at least about 15-20% changes being preferred and at least about 30% changes being particularly preferred.

These sequences can be used in a number of ways. In a preferred embodiment, one, some or all of the optimized protein sequences are constructed into designed proteins, as show with step 58 of FIG. 2. Thereafter, the protein sequences can be tested, as shown with step 60 of the FIG. 2. Generally, this can be done in one of two ways.

In a preferred embodiment, the designed proteins are chemically synthesized as is known in the art. This is particularly useful when the designed proteins are short, preferably less than 150 amino acids in length, with less than 100 amino acids being preferred, and less than 50 amino acids being particularly preferred, although as is known in the art, longer proteins can be made chemically or enzymatically.

In a preferred embodiment, particularly for longer proteins or proteins for which large samples are desired, the optimized sequence is used to create a nucleic acid such as DNA which encodes the optimized sequence and which can then be cloned into a host cell and expressed. Thus, nucleic acids, and particularly DNA, can be made which encodes each optimized protein sequence. This is done using well known procedures. The choice of codons, suitable expression vectors and suitable host cells will vary depending on a number of factors, and can be easily optimized as needed.

Once made, the designed proteins are experimentally evaluated and tested for structure, function and stability, as required. This will be done as is known in the art, and will depend in part on the original protein from which the protein backbone structure was taken. Preferably, the designed proteins are more stable than the known protein that was used as the starting point, although in some cases, if some constraints are placed on the methods, the designed protein may be less stable. Thus, for example, it is possible to fix certain residues for altered biological activity and find the most stable sequence, but it may still be less stable than the wild type protein. Stable in this context means that the new protein retains either biological activity or conformation past the point at which the parent molecule did. Stability includes, but is not limited to, thermal stability, i.e. an increase in the temperature at which reversible or irreversible denaturing starts to occur; proteolytic stability, i.e. a decrease in the amount of protein which is irreversibly cleaved in the presence of a particular protease (including autolysis); stability to alterations in pH or oxidative conditions; chelator stability; stability to metal ions; stability to solvents such as organic solvents, surfactants, formulation chemicals; etc.

In a preferred embodiment, the modeled proteins are at least about 5% more stable than the original protein, with at least about 10% being preferred and at least about 20-50% being especially preferred.

The results of the testing operations may be computationally assessed, as shown with step 62 of FIG. 2. An assessment module 38 may be used in this operation. That is, computer code may be prepared to analyze the test data with respect to any number of matrixes.

At this processing juncture, if the protein is selected (the yes branch at block 64) then the protein is utilized (step 66), as discussed below. If a protein is not selected, the accumulated information may be used to alter the ranking module 34, and/or step 56 is repeated and more sequences are searched.

In a preferred embodiment, the experimental results are used for design feedback and design optimization.

Once made, the proteins of the invention find use in a wide variety of applications, as will be appreciated by those in the art, ranging from industrial to pharmacological uses, depending on the protein. Thus, for example, proteins and enzymes exhibiting increased thermal stability may be used in industrial processes that are frequently run at elevated temperatures, for example carbohydrate processing (including saccharification and liquifaction of starch to produce high fructose corn syrup and other sweetners), protein processing (for example the use of proteases in laundry detergents, food processing, feed stock processing, baking, etc.), etc. Similarly, the methods of the present invention allow the generation of useful pharmaceutical proteins, such as analogs of known proteinaceous drugs which are more thermostable, less proteolytically sensitive, or contain other desirable changes.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are explicitly incorporated by reference in their entirety.

### EXAMPLES

### Example 1

Benchmark Design Cases

To demonstrate the increased power of split DEE over standard Goldstein DEE, the present study presents results for five challenging design problems described in Table I. Case 1 represent the design of all 25 non-glycine residues (5, 14, 21, 27, 29, 31, 35, 37, 38, 39, 41, 46, 50, 55, 56, 63, 70, 72, 74, 80, 82, 84, 92, 96, 98) in the core of plastocyanin (PDB code 2pcy). Case 2 involves all 34 core positions on a novel repeating backbone based on the leucine rich repeat motif; the 17 residues in each of two repeats have linked, but unspecified amino acid identiities. Case 3 represents the full core design of the variable domains of the light and heavy chains of catalytic antibody 48G7 FAB (PDB code 1gaf). This corresponds to residues (2, 4, 6, 19, 21, 25, 29, 33, 36-38, 44, 46-48, 55, 58, 62, 71, 73, 75, 78, 82, 84-87, 89, 90, 95-98, 102, 104) of chain L and residues (4, 6, 18, 20, 24, 32, 34-39, 45, 47, 48, 50, 51, 53, 61, 64, 68, 70, 72, 77, 79, 81, 83, 86, 90, 92-95, 97, 98, 103, 104, 108, 110) of chain H. Case 4 involves the design of all 10 non-glycine core residues (3, 5, 7, 20, 26, 30, 34, 39, 52, 54) and all 15 boundary residues (1, 11, 12, 16, 18, 23, 25, 27, 29, 33, 37, 43, 45, 50, 56) of the β1 domain of Protein G (PDB code 1 pga). Case 5 represent the design of all 27 non-glycine surfaces residues (2, 4, 6, 8, 10, 13, 15, 17, 19, 21, 22, 24, 28, 31, 32, 35, 36, 40, 42, 44, 46, 47, 48, 49, 51, 53, 55) of the β1 domain of Protein G. The benchmark calculations were performed on 16 Power3 processors of an IBM SP3 running at 375 MHz. The CPU minutes for the benchmark design cases is shown in Table 2.

TABLE 1
Benchmark Design Cases

| Case | Description | Type | Residues | Rotamers | Conformations |
|---|---|---|---|---|---|
| 1 | Plastocyanin | Core | 25 | 1716 | 1.7 × 10³⁸ |
| 2 | Novel | Linked | 34 | 674 | 8.4 × 10³⁹ |
| | backbone | Core | | | |
| 3 | Catalytic | CORE | 74 | 4919 | 4.7 × 10¹²⁸ |
| | antibody | | | | |
| 4 | β1 of Protein | Core/ | 25 | 4295 | 4.0 × 10⁵³ |
| | G | boundary | | | |
| 5 | β1 of Protein | Surface | 27 | 4842 | 4.9 × 10⁶⁰ |
| | G | | | | |

TABLE 2
CPU minutes for the benchmark design cases.

| | | | Remaining |
|---|---|---|---|
| Case | Method | Time (minutes) | Conformations |
| 1 | DEE s2mb | 334 | 7 × 10¹⁴ |
| | DEE s2 | 150 | 2 × 10¹¹ |
| | DEE s2 bound flags | 22 | 1 |
| | DEE s2 split flags | 46 | 1 |
| | HERO | 13 | 1 |
| 2 | DEE s2mb | 250 | 1 × 10¹⁸ |
| | DEE s2 | 210 | 1 × 10¹⁸ |
| | DEE s2 bound flags | 23 | 1 |
| | DEE s2 split flags | 167 | 3 × 10¹⁶ |
| | HERO | 7 | 1 |
| 3 | DEE s2mb | 984 | 3 × 10⁸ |
| | DEE s2 | 687 | 1 |
| | DEE s2 bound flags | 663 | 1 |
| | DEE s2 split flags | 299 | 1 |
| | HERO | 359 | 1 |
| 4 | DEE s2mb | 1449 | 2 × 10³⁵ |
| | DEE s2 | 1333 | 1 × 10³⁵ |
| | DEE s2 bound flags | 1688 | 1 × 10³⁵ |
| | DEE s2 split flags | 875 | 9 × 10¹⁹ |
| | HERO | 476 | 1 |
| 5 | DEE s2mb | 292 | 3 × 10¹⁶ |
| | DEE s2 | 129 | 1 |
| | DEE s2 bound flags | 72 | 1 |
| | DEE s2 split flags | 46 | 1 |
| | HERO | 35 | 1 |

The HERO Algorithm

Using a rotameric description of conformational space, the Side Chain Placement Problem of Homology Modeling (Desmet, J., et al., (1992) Nature, 365:539) and the Sequence Selection Problem of Protein Design (Dahiyat, B. I., and Mayo, S L. (1996) Prot. Sci., 5:895) can both be described as the following combinatorial optimization problem:

Choose the single rotamer for each residue position that minimizes the sum of the pairwise interaction energies between rotamers at all positions.

The problem may also be recast as the Flight Pricing Problem:

For a set of cities each containing multiple airports, choose the airport for each city that minimizes the cost of visiting every city from every other city.

and as The Belief Network Problem (Pearl, J. (1988) “Probabilistic Reasoning in Intelligent Systems”, Morgan-Kauffman):

For a graph with nodes representing conditional probabilities and edges representing dependencies between these probabilities, determine the values at the nodes that maximize the sum of these conditional probabilities.

and the Spin Glass Problem (Mezard, G., et al., (1987), “Spin Glass Theory and Beyond”, World Scientific):

For a graph with nodes representing spin states and edges representing coupling between spin states at neighboring nodes, find the set of spins that correspond to the lowest energy ground state of the system.

The common mathematical structure in all of these problems is that the system is defined by a set of candidate solutions at each of a number of nodes. Each candidate solution is described in terms of a self-energy (possibly zero) and a set of pairwise interaction energies (possibly zero) with candidate solutions at other nodes. The goal is to find a list of unique candidate solutions (one for each node) that produces the global optimum of a specified quantity which is based on these self-and pairwise energies. The HERO algorithm is an exact search algorithm for solving any problem with this structure.

The HERO Algorithm

At convergence, this algorithm identifies the single rotamer at each position that belongs to the global minimum energy conformation. HERO may be run using simple split singles DEE or split singles DEE with split flags. The following cycle is an example of HERO run with split singles DEE. This cycle is repeated until the global minimum energy conformation (GMEC) is identified by eliminating all but one rotamer at each position:

1) Iterative simple Goldstein singles DEE (T=1) until no further eliminations;

2) Iterative simple split singles DEE (s=1) until no further eliminations;

3) Split single DEE (s>1) with or without magic bullet metric once for each rotamer;

4) Apply singles bounding criteria to eliminate rotamers whose bounding energy E_{bound} is greater than E_{low}, the lowest known energy of a valid conformation obtained from the Monte Carlo searches;

5) Alternate sequentially between the following, applying one during each cycle:

a) Magic bullet DEE Goldstein doubles calculation (T=1) to flag dead ending pairs;

b) Monte Carlo search to find a low energy of a valid conformation E_{low} ;

c) Apply doubles bounding criteria to flag pairs whose bounding energy E_{bound} is greater than E_{low};

d) Goldstein DEE doubles calculation (T=1) to flag dead ending pairs; and,

e) Unification of any uniquely defined positions, followed by unification of the two residues with the highest fraction of dead ending pairs, followed by restoration of all flags for the new super-residues;

6) return to 1.

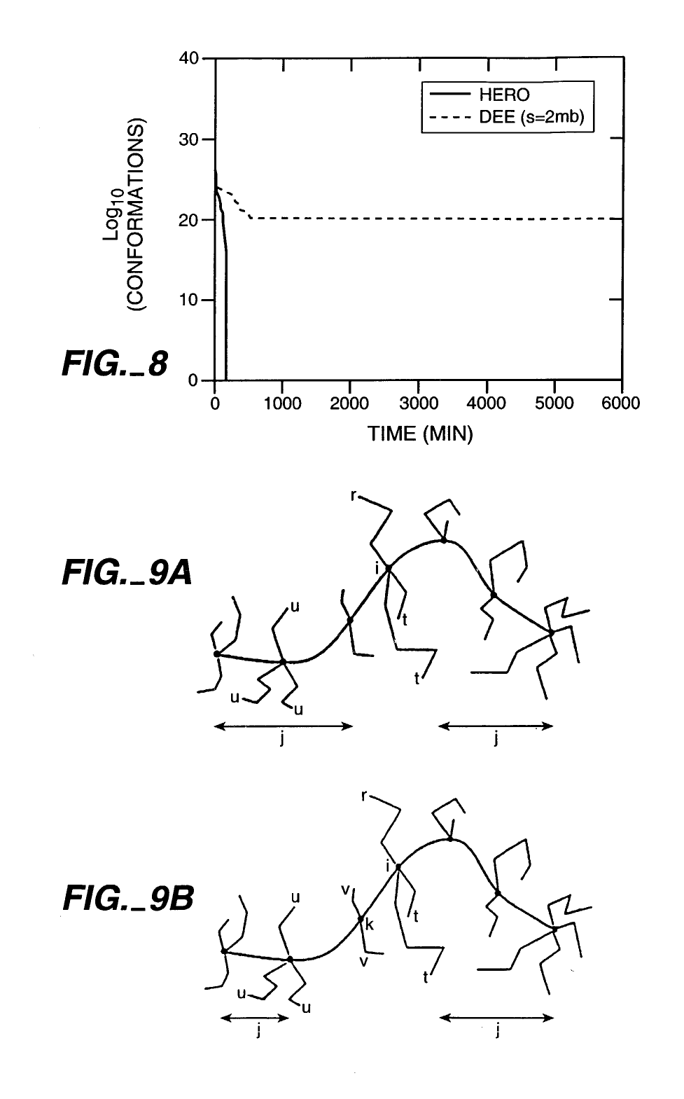

A sample calculation comparing the performance of HERO, run using simple split DEE, to the previous state-of-the-art DEE algorithm is shown in FIG. 8. The initial number of conformations is 8.4×1039 and DEE (s=2_{mb}) fails to reduce the number of conformations below 1.0×10²⁰ after more than 6000 minutes, while HERO converges to the unique minimum conformation in 167 minutes.

### Example 3

Physical Model Dependence

The potential function has ben previously described (see Dahiyat, B. I. and Mayo, S. L. (1996) Protein Sci., 5:895-903; Dahiyat, B. I. and Mayo, S. L., (1997) Science, 278:82-87) and incorporates terms for van der Waals interactions, hydrogen bonds, Coulombic interations and solvation. The backbone-dependent rotamer libraries are based on the mean values from the Dunbrack and Karplus library (*reference) with expansion of the χ₁ and χ₂ angles for aromatic residues, the χ₁ angle for hydrophobic residues and no expansion for polar residues. Canonical values for χ₃ and χ₄ angles are used for amino acids E, Q, K and R. Residues are classified into core, boundary or surface positions (as described herein). Core residue identities are selected from among the amino acids A, V, L, I, F, Y and W, while surface residue identities are selected form among A, S, T, D, N, H, E, Q, K and R. Boundary residue identities are chosen from the union of these sets.

An example of the dependence of the optimization performance on the underlying physical model is shown in FIG. 15. This case is a full sequence design of the 56 positions in the β1 domain of Protein G. Three of these positions are preset to glycine (position 38 has a positive phi angle and functions as a C-cap for the alpha helix; positions 9 and 41 are sterically constrained core positions). The remaining positions are divided into core, boundary and surface regions with the allowed amino acid identities at each of the 53 positions constrained to preserve the binary pattern of the wild-type sequence. The resulting combinatorial complexity is 10¹¹² conformations with 7775 initial rotamers after applying high-energy threshold reduction (HETR; DeMaeyer, M., et al., (1997) Fold. Des., 2:53-66) to eliminate rotamers that clash with the backbone. A HERO run with “standard” potential function and rotamer library fails to converge after more than 1000 minutes. Optimization with a potential function modified to emphasize one-body terms reaches the GMEC in 20 minutes.

In addition to the potential function component of the physical model, care must be taken with respect to the rotamer library. Previous computational work using surprisingly small rotamer libraries (approximately 67 rotamers per residue position) showed large, full sequence design problems to be tractable (Looger, L. L. and Hellinga, H. W. (2001) J. Mol. Biol., 307:429-445). For the full sequence design of Protein G, the average number of rotamers per residue position is 147. Using an unexpanded rotamer library and aggressive HETR, the average number of rotamers per position can be reduced to 70 (10⁸⁰ conformations for 3705 rotamers). Obtaining the GMEC for the resulting problem using the standard potential function requires 28 minutes. These results strikingly illustrate the dependence of algorithm performance on both the potential function and the rotamer library. Clearly, search algorithm performance cannot be meaningfully ascertained on models of uncertain biological validity.

We claim:

1. A method executed by a computer under the control of a program, said computer including a memory for storing said program, said method comprising the steps of:
(A) receiving a protein backbone structure with variable residue positions;
(B) establishing a group of potential rotamers for each of said variable residue positions, wherein at least one variable residue position has rotamers from at least two different amino acid side chains; and,
(C) analyzing the interaction of each of said rotamers with all or part of the remainder of said protein structure to generate a set of optimized protein sequences, wherein said analyzing step includes a split singles Dead End Elimination (DEE) with a split flags computation.

2. A method according claims 1 wherein said analyzing step includes the use of at least one scoring function.

3. A method according to claim 1 wherein said analyzing step includes a Hybrid Exact Rotamer Optimization (HERO) step.

4. A method according to claim 1 wherein said set of optimized protein sequences comprises the globally optimal protein sequence.

5. A method according to claim 1 further comprising testing at least one member of said set to produce experimental results.

6. A method according to claim 4 further comprising:
(D) generating a rank ordered list of additional optimal sequences from said global optimal protein sequence.

7. A method according to claim 6 wherein said generating includes the use of a Monte Carlo search.

8. A method according to claim 1 wherein said analyzing step comprises a Monte Carlo computation.

9. An optimized protein sequence generated by the method of claim 1.

10. A nucleic acid sequence encoding a protein sequence according to claim 9.

11. An expression vector comprising the nucleic acid of claim 10.

12. A host cell comprising the nucleic acid of claim 10.

13. A computer readable memory to direct a computer to function in a specified manner, comprising:
a side chain module to correlate a group of potential rotamers for residue positions of a protein backbone model;
a ranking module to analyze the interaction of each of said rotamers with all or part of the remainder of said protein to generate a set of optimized protein sequences wherein said analysis includes a split singles DEE with a split flags computation.

14. A computer readable memory according to claim 13 wherein said ranking module includes a HERO computation step.

15. A method executed by a computer under the control of a program, said computer including a memory for storing said program, said method comprising the steps of:
(A) receiving a protein backbone structure with variable residue positions;
(B) establishing a group of potential rotamers for each of said variable residue positions, wherein the group of potential rotamers for at least one of said variable residue position has a rotamer selected from each of at least two different amino acid side chains; and
(C) analyzing the interaction of each of said rotamers with all or part of the remainder of said protein backbone structure to generate a set of optimized protein sequences, wherein said analyzing step includes a split singles DEE with split flags computation.
